(19) United States
(12) Patent Application Publication
(10) Pub. No.: US 2021/0177495 A1
(43) Pub. Date: Jun. 17, 2021

(54) ELECTROSURGICAL INSTRUMENTS FOR SEALING AND DISSECTION

(71) Applicant: Intuitive Surgical Operations Inc., Sunnyvale, CA (US)

(72) Inventors: Adam Ross, Prospect, CT (US); Robert Reid, Fairfield, CT (US)

(21) Appl. No.: 17/084,981

(22) Filed: Oct. 30, 2020

Related U.S. Application Data
(60) Provisional application No. 62/947,307, filed on Dec. 12, 2019.
(60) Provisional application No. 62/947,263, filed on Dec. 12, 2019.

(51) Int. Cl. A61B 18/14; A61B 34/30

(52) U.S. Cl. CPC A61B 18/1445; A61B 2018/00601; A61B 2018/1452; A61B 34/30

(57) ABSTRACT

A surgical instrument comprises first and second jaws movable relative to each other between open and closed positions, a cutting electrode with a cutting surface for tissue dissection and at least one sealing electrode for sealing or coagulating tissue. The cutting electrode includes one or more insulation layers for securing the cutting electrode to one of the jaws and for protecting the jaws from the heat and energy generated at the cutting surface during operation. An actuator mechanism is coupled to the first and second jaws for moving the jaws between the open and closed positions. At least one portion of the actuator mechanism comprises a conductive pathway for electrically coupling the cutting and/or sealing electrode(s) to a source of energy. Thus, the mechanical components of the actuator mechanism include electrically conductive pathways to reduce the number of conductors extending through the device, thereby providing a more compact and maneuverable instrument.

### CROSS-REFERENCE TO RELATED APPLICATIONS

This application claims the benefit of U.S. Provisional Application Ser. No. 62/947,307, filed Dec. 12, 2019 and U.S. Provisional Application Ser. No. 62/947,263, filed Dec. 12, 2019, the entire disclosures of which are incorporated herein by reference for all purposes.

### BACKGROUND

The field of the present disclosure relates to medical instruments, and more particularly to electrosurgical instruments with opposing jaws that apply sufficient gripping forces to handle, seal, staple and/or cut tissue and vessels of varying size and diameter.

Minimally invasive medical techniques are intended to reduce the amount of extraneous tissue that is damaged during diagnostic or surgical procedures, thereby reducing patient recovery time, discomfort, and deleterious side effects. One effect of minimally invasive surgery, for example, is reduced post-operative hospital recovery times. The average hospital stay for a standard open surgery is typically significantly longer than the average stay for an analogous minimally invasive surgery (MIS). Thus, increased use of MIS could save millions of dollars in hospital costs each year. While many of the surgeries performed each year in the United States could potentially be performed in a minimally invasive manner, only a portion of the current surgeries uses these advantageous techniques due to limitations in minimally invasive surgical instruments and the additional surgical training involved in mastering them.

Improved surgical instruments such as tissue access, navigation, dissection and sealing instruments have enabled MIS to redefine the field of surgery. These instruments allow surgeries and diagnostic procedures to be performed with reduced trauma to the patient. A common form of minimally invasive surgery is endoscopy, and a common form of endoscopy is laparoscopy, which is minimally invasive inspection and surgery inside the abdominal cavity. In standard laparoscopic surgery, a patient's abdomen is insufflated with gas, and cannula sleeves are passed through small (approximately one-half inch or less) incisions to provide entry ports for laparoscopic instruments.

Laparoscopic surgical instruments generally include an endoscope (e.g., laparoscope) for viewing the surgical field and tools for working at the surgical site. The working tools are typically similar to those used in conventional (open) surgery, except that the working end or end effector of each tool is separated from its handle by an extension tube (also known as, e.g., an instrument shaft or a main shaft). The end effector can include, for example, a clamp, grasper, scissor, stapler, cautery tool, linear cutter, or needle holder.

To perform surgical procedures, the surgeon passes working tools through cannula sleeves to an internal surgical site and manipulates them from outside the abdomen. The surgeon views the procedure from a monitor that displays an image of the surgical site taken from the endoscope. Similar endoscopic techniques are employed in, for example, arthroscopy, retroperitoneoscopy, pelviscopy, nephroscopy, cystoscopy, cistemoscopy, sinoscopy, hysteroscopy, urethroscopy, and the like.

Minimally invasive telesurgical robotic systems are being developed to increase a surgeon's dexterity when working on an internal surgical site, as well as to allow a surgeon to operate on a patient from a remote location (outside the sterile field). In a telesurgery system, the surgeon is often provided with an image of the surgical site at a control console. While viewing a three dimensional image of the surgical site on a suitable viewer or display, the surgeon performs the surgical procedures on the patient by manipulating master input or control devices of the control console, which in turn control motion of the servo-mechanically operated slave instruments.

The servomechanism used for telesurgery will often accept input from two master controllers (one for each of the surgeon's hands) and may include two or more robotic arms on each of which a surgical instrument is mounted. Operative communication between master controllers and associated robotic arm and instrument assemblies is typically achieved through a control system. The control system typically includes at least one processor that relays input commands from the master controllers to the associated robotic arm and instrument assemblies and back from the instrument and arm assemblies to the associated master controllers in the case of, for example, force feedback or the like. One example of a robotic surgical system is the DA VINCI™ system commercialized by Intuitive Surgical, Inc. of Sunnyvale, Calif.

A variety of structural arrangements have been used to support the surgical instrument at the surgical site during robotic surgery. The driven linkage or “slave” is often called a robotic surgical manipulator, and exemplary linkage arrangements for use as a robotic surgical manipulator during minimally invasive robotic surgery are described in U.S. Pat. No. 7,594,912 (filed Sep. 30, 2004), U.S. Pat. No. 6,758,843 (filed Apr. 26, 2002), U.S. Pat. No. 6,246,200 (filed Aug. 3, 1999), and U.S. Pat. No. 5,800,423 (filed Jul. 20, 1995), the full disclosures of which are incorporated herein by reference in their entirety for all purposes. These linkages often manipulate an instrument holder to which an instrument having a shaft is mounted. Such a manipulator structure can include a parallelogram linkage portion that generates motion of the instrument holder that is limited to rotation about a pitch axis that intersects a remote center of manipulation located along the length of the instrument shaft. Such a manipulator structure can also include a yaw joint that generates motion of the instrument holder that is limited to rotation about a yaw axis that is perpendicular to the pitch axis and that also intersects the remote center of manipulation. By aligning the remote center of manipulation with the incision point to the internal surgical site (for example, with a trocar or cannula at an abdominal wall during laparoscopic surgery), an end effector of the surgical instrument can be positioned safely by moving the proximal end of the shaft using the manipulator linkage without imposing potentially hazardous forces against the abdominal wall. Alternative manipulator structures are described, for example, in U.S. Pat. No. 6,702,805 (filed Nov. 9, 2000), U.S. Pat. No. 6,676,669 (filed Jan. 16, 2002), U.S. Pat. No. 5,855,583 (filed Nov. 22, 1996), U.S. Pat. No. 5,808,665 (filed Sep. 9, 1996), U.S. Pat. No. 5,445,166 (filed Apr. 6, 1994), and U.S. Pat. No. 5,184,601 (filed Aug. 5, 1991), the full disclosures of which are incorporated herein by reference in their entirety for all purposes.

During the surgical procedure, the telesurgical system can provide mechanical actuation and control of a variety of surgical instruments or tools having end effectors that perform various functions for the surgeon, for example, holding or driving a needle, grasping a blood vessel, dissecting tissue, or the like, in response to manipulation of the master input devices. Manipulation and control of these end effectors is a particularly beneficial aspect of robotic surgical systems. Such mechanisms should be appropriately sized for use in a minimally invasive procedure and relatively simple in design to reduce possible points of failure. In addition, such mechanisms should provide an adequate range of motion to allow the end effector to be manipulated in a wide variety of positions.

Various surgical instruments are configured to apply electrical energy to tissue during surgical procedures. For example, a surgical instrument may be configured to seal, bond, ablate, dissect, fulgurate, etc. tissue through the application of an electrical current. In some cases, the body of a patient is held at a ground (e.g., zero) electrical potential, while a portion of the surgical instrument is brought to a different electrical potential (e.g., by an operator command to the surgical system) to deliver electrical energy to the surgical site. In other instances, surgical instruments may be capable of conducting bipolar energy through grasped tissue by having one jaw member having a first electrical potential, and a second jaw member having a second electrical potential.

Electrosurgical instruments may include cutting elements for dissecting tissue by creating a high density energy surface on the cutting element. The high density energy creates heat, which vaporizes tissue in contact with the electrode, resulting in tissue being transected along the surface of the electrode. The high density energy and excess heat created at and around the electrode, however, can cause interference with, or damage to, other components of the surgical instrument or other objects at the surgical site, such as staples and the like.

Certain electrosurgical instruments may also include bipolar coagulation electrodes. To supply electrical energy to the coagulation electrodes and/or to the cutting electrode, electrical conductors are typically extended through the instrument shaft to each of the electrodes. These electrical conductors require insulation components to insulate the conductors from the rest of the instrument. The conductors and the insulating components, however, increase the complexity of the instrument design and consume space within the shaft and the end effector, which may increase the overall size of the surgical instrument.

Accordingly, while the new telesurgical systems and devices have proven highly effective and advantageous, still further improvements would be desirable. In general, it would be desirable to provide improved electrosurgical instruments that are more compact and maneuverable to enhance the efficiency and ease of use of minimally invasive systems. In addition, it would be beneficial to provide improved electrode designs that provide optimal performance, while adequately protecting the components of the instrument and/or other objects at the surgical site.

### SUMMARY

The following presents a simplified summary of the claimed subject matter in order to provide a basic understanding of some aspects of the claimed subject matter. This summary is not an extensive overview of the claimed subject matter. It is intended to neither identify key or critical elements of the claimed subject matter nor delineate the scope of the claimed subject matter. Its sole purpose is to present some concepts of the claimed subject matter in a simplified form as a prelude to the more detailed description that is presented later.

A surgical instrument comprises first and second jaws movable relative to each other between open and closed positions, a cutting electrode with a cutting surface for tissue dissection and at least one sealing electrode for sealing or coagulating tissue. The cutting electrode includes one or more insulation layers for securing the cutting electrode to one of the jaws and for protecting the jaws from the heat and energy generated at the cutting surface during operation. An actuator mechanism is coupled to the first and second jaws for moving the jaws between the open and closed positions. At least one portion of the actuator mechanism comprises a conductive pathway for electrically coupling the cutting and/or sealing electrode(s) to a source of energy. Thus, one or more of the mechanical components of the actuator mechanism (whose primary function is to cooperate and control the movement of the first and second jaws between the open and closed positions) have a secondary function of providing at least one electrically conductive pathway through which the cutting and/or sealing electrode may be coupled to the source of energy, thereby providing a more compact and maneuverable instrument.

In one embodiment, the actuator mechanism comprises a cam slot in the first jaw and a pin positioned within the cam slot such that translation of the pin through the slot rotates the jaws between the open and closed positions. The pin comprises a conductive material and is configured for electrical coupling to the source of energy, which may be, for example, coupled to the proximal end of the instrument. In certain embodiments, at least one surface of the cam slot comprises a conductive material such that contact between the pin and the conductive surface electrically couples the electrode(s) to the source of energy. In an exemplary embodiment, the cam slot is designed such that translation of the pin through the slot provides electrical current to the electrode(s) at a specific point in the pathway of the pin through the slot (e.g., when the jaws are in a closed or partially closed position).

The actuator assembly may further comprise a drive rod for translating the pin through the slot. The drive rod comprises a conductive pathway electrically coupling the pin with the source of energy. In a preferred embodiment, the conductive pathway comprises a conductor that extends through a drive tube. The conductor has a proximal end configured for coupling to the source of energy and a distal end electrically coupled to the pin.

In certain embodiments, the surgical instrument includes a second slot in the second jaw. The pin is positioned within the second slot such that translation of the pin through the first and second slots rotates the jaws between the open and closed positions. The second slot includes insulating material between the pin and the second slot to electrically isolate the pin from the second jaw. This ensures that the second jaw is electrically isolated from the conductive pathway to the electrode(s) in the first jaw.

In another embodiment, the instrument includes a coupling member coupling the elongate shaft with the first and second jaws. The coupling member includes one or more conductive members or surfaces configured for coupling to the source of energy to provide electrical energy to the electrode. In certain embodiment, the surgical instrument may comprise a second electrode on the second jaw. In this configuration, the second electrode is electrically coupled to the conductive member(s) or surface(s) within the coupling member and the first electrode is electrically coupled to the cam slot. This provides electrical energy to both the first and second electrodes with mechanical components of the instrument, thereby reducing the requirement for additional conductors and insulating components within the instrument.

In certain embodiments, the coupling member comprises an articulation mechanism configured to articulate the first and second jaws relative to the elongate shaft. The articulation mechanism may comprise a wrist assembly that comprises a conductive material in at least one portion of the wrist assembly. The instrument may further comprise an insulating sheath disposed around the wrist assembly to electrically isolate the wrist assembly from the surrounding environment.

In another aspect of the present disclosure, a surgical instrument comprises an elongate shaft coupled to an end effector having opposing jaws that open and close relative to each other. An electrode has a cutting surface for tissue dissection and is coupled to one of the jaws such that the cutting surface extends away from the jaw. A first insulating layer covers a first portion of the electrode and has an attachment structure for attaching the electrode to the jaw. A second insulating layer covers a second portion of the electrode such that the cutting surface remains exposed. The insulating layers serve to both attach the electrode to the jaw and to protect the instrument components from the high density energy generated on the cutting surface of the electrode.

The first insulating layer preferably comprises a material that has high temperature resistance, electrical isolation and sufficient rigidity to provide a stable mechanical attachment of the electrode to the first jaw, such as plastic, ceramic, or any other moldable insulating material. The second insulating layer preferably comprises a material with high dielectric strength and high temperature resistance to prevent damage to the insulation due to the high temperatures created in operation. In a preferred embodiment, the second insulating material will comprise a hydrophobic material that also has non-stick properties and has a relatively high comparative tracking index (CTI), such as silicone rubber or a similar material. These properties help deter the incidence of arc tracking across the surface of the insulating layer from the high voltages required for operation of the cutting electrode.

In certain embodiments, the first jaw comprises an opening and the attachment structure comprises a post extending from the first insulating layer through the opening. After passing through the opening, the post is deformed to secure the post within the opening of the first jaw, either through cold forming or thermoplastic staking (i.e., heat staking). This configuration effectively secures the electrode to the jaw.

In another embodiment, the first jaw and the first insulating layer each comprise a pivot hole for receiving a pivot pin therethrough. The pivot pin is configured to allow the first jaw to pivot relative to the second jaw. By forming the first insulating layer such that its pivot hole aligns with the pivot hole in the first jaw, this provides an additional form of attachment for securing the electrode to the first jaw and allows the entire jaw and electrode assembly to rotate with respect to the second jaw.

In other embodiments, the electrode may comprise a number of openings and the insulating layers may be formed through the openings to further secure the insulating layers to the electrode. In one such embodiment, the electrode comprises a first row of holes extending along its longitudinal axis and the first insulating layer is formed such that it extends through the first row of holes. The electrode may further comprise second row of holes and the second insulating layer is formed such that it extends through the second row of holes.

In an exemplary embodiment, the electrode is a cutting electrode configured for dissecting tissue. The first jaw further comprises one or more sealing electrodes configured for sealing tissue, preferably spaced on either side of the cutting electrode to seal tissue on either side of the dissection. In certain embodiments, the second jaw also comprises one or more sealing electrodes and the instrument is configured to transmit electric current from the sealing electrodes on the first jaw, through tissue, to the sealing electrodes in the second jaw. In an exemplary embodiment, one or both of the jaws comprises one or more spacers extending outward beyond the tissue contacting surfaces of the sealing electrodes to space the sealing electrodes on the first jaw from the sealing electrodes on the second jaw when the first and second jaws are in the closed or partially closed position.

In another aspect of the invention, a method for manufacturing a surgical instrument comprises attaching a first insulating layer to a portion of an electrode and securing the insulating layer to a jaw on the end effector of the surgical instrument. A second insulating layer is attached to another portion of the electrode such that a cutting surface on the electrode is exposed. The insulating layers protect the other components of the instrument and the surrounding environment from the high density energy formed on the cutting surface during use. The first insulating layer serves to both protect the instrument from this energy and to secure the electrode to the jaw.

In one embodiment, the electrode comprises first and second set of holes. The first insulating layer is injected molded through the first set of holes and the second insulating layer is injected molded through the second set of holes. This ensures that the first and second insulating layers remain securely attached to the electrode.

In certain embodiments, the first insulating layer comprises a post on a surface opposite the electrode and the jaw includes an opening or hole. The post is passed through the opening and deformed such that the post is secured to the hole. In an exemplary embodiment, the post is deformed through cold forming or thermoplastic staking (e.g., heat staking).

The first insulating layer and the jaw may further include pivot pin holes. The method includes aligning the pivot pin holes with each other and advancing a pivot pin through the holes to further secure the first insulating layer to the jaw and to allow for rotation of both components relative to a second jaw on the end effector.

In another aspect of the invention, a surgical instrument comprises first and second jaws movable relative to each other between open and closed positions and a cutting electrode coupled to the first jaw and having a cutting surface. The instrument further comprises one or more sealing electrode(s) on the first jaw having tissue contacting surfaces and residing on first and second sides of the cutting electrode. The cutting surface of the cutting electrode extends from the first jaw beyond the tissue contacting surfaces of the sealing electrodes. The instrument further includes one or more sealing electrodes on the second jaw having tissue contacting surfaces opposite the tissue contacting surfaces of the first sealing electrodes. This configuration allows for bipolar cutting and coagulation operations to be performed such that a bipolar seal is provided on either side of the line of dissection.

In certain embodiments, one or both of the first and second jaws include spacers extending therefrom such that the first and second sealing electrodes are spaced from each other when the first and second jaws are in the closed position. The jaws preferably each comprise a cam slot and a pin is positioned within the cam slots. An actuation mechanism is coupled to the pin to translate the pin through the cam slots, thereby rotating the jaws between the open and closed positions. In an exemplary embodiment, the actuator includes a control device of a robotic telesurgical system that may, for example, allow for mechanical actuation and control of the surgical instrument to perform a variety of functions, such as grasping a blood vessel, sealing and/or dissecting tissue, or the like, in response to manipulation of master input devices located remotely from the surgical instrument.

In one embodiment, at least one of the cam slots has a non-linear shape such that at least one of the jaws applies a grip force that is substantially proportional to a force applied to the pin to translate the pin through at least one portion of the slots (i.e., the ratio between the input force and the resulting output force remains substantially the same as the pin travels through at least one portion of the slots). This design provides a constant mechanical advantage between the force applied to the pin and the force applied by the jaws to tissue held therebetween, thereby allowing a user (or a robotic system) to more easily regulate the forces applied to tissue by the jaws.

In addition, this design allows for a substantially constant grip force to be applied by the jaws regardless of the angle between the jaws. Therefore, the jaws may apply substantially the same amount of grip force against, for example, a larger vessel or tissue portion that requires the jaws to remain further open (e.g., greater than 20% of the fully open jaw configuration).

It is to be understood that both the foregoing general description and the following detailed description are exemplary and explanatory only and are not restrictive of the disclosure. Additional features of the disclosure will be set forth in part in the description which follows or may be learned by practice of the disclosure.

### BRIEF DESCRIPTION OF THE DRAWINGS

The accompanying drawings, which are incorporated in and constitute a part of this specification, illustrate several embodiments of the disclosure and together with the description serve to explain the principles of the disclosure.

FIG. 1 is a side view of the distal end portion of a surgical instrument in accordance with an illustrative embodiment of this disclosure with the jaws of the end effector in the closed position;

FIG. 2 is a perspective view of the distal end portion of the surgical instrument of FIG. 1;

FIG. 3 is a side view of the end effector of the surgical instrument of FIG. 1 with the jaws of the end effector in the open position;

FIG. 4 is a side view of a distal portion of the end effector of the surgical instrument of FIG. 1 with certain parts cut-a-way;

FIG. 4A is a partial cut-a-way view of an alternative embodiment of an end effector of the surgical instrument of FIG. 1 in which the wrist assembly provides an electrical pathway to energize the stationary jaw;

FIG. 5 is a top-down view of the end effector of the surgical instrument of FIG. 1;

FIG. 6 is a cross-sectional bottom view of the movable jaw of the end effector of the surgical instrument of FIG. 1 showing the cutting electrode between the sealing surfaces;

FIG. 7 is a cross-sectional view of the surgical instrument of FIG. 1 with the jaws of the end effector in the closed position;

FIG. 8 is a side view of a drive assembly of an end effector of the surgical instrument of FIG. 1;

FIG. 9 is a side view of an end effector of a surgical instrument with a non-linear cam slot according to certain embodiments of the present disclosure

FIG. 10 is a side view of another embodiment of an end effector of a surgical instrument with a compound cam slot according to certain embodiments of the present disclosure;

FIG. 11 is a side view of an electrode blank for use in preparing an electrode assembly for use in a surgical instrument according to the present disclosure;

FIG. 12 is a perspective view of the electrode blank of FIG. 11 after being coined;

FIG. 13 is a side view of the electrode of FIG. 12 having a first insulator layer molded thereon;

FIG. 14 is a bottom view of the electrode and insulator layer of FIG. 13;

FIG. 15 is a side view of the electrode and insulator layer of FIG. 14 showing a conductor wire attached to the tab of the electrode thereto;

FIG. 16 is a bottom view of the electrode, insulator layer, and conductor wire of FIG. 15;

FIG. 17 is a side view of the wired electrode of FIG. 15 having a second insulator layer overmolded thereon;

FIG. 17A is a partial cross-sectional view of the electrode of FIG. 17 taken along lines 17A-17A;

FIG. 18 is a perspective view of the installation of a cutting electrode assembly into the jaw of an end effector of a surgical instrument in accordance with an illustrative embodiment of this disclosure;

FIG. 19 is a perspective view of the jaw of an end effector of a surgical instrument in accordance with an illustrative embodiment of this disclosure having a cutting electrode assembly installed therein;

FIG. 20A is a perspective view of a portion of an end effector of a surgical instrument in accordance with an illustrative embodiment of this disclosure having an attachment structure, e.g., a post, that has not yet been heat staked;

FIG. 20B is a perspective view of the surgical instrument of FIG. 20A after the post has been heat staked;

FIG. 21 is a bottom view of a jaw of an end effector of a surgical instrument in accordance with an illustrative embodiment of this disclosure;

FIG. 22 is a side view of a cutting electrode assembled into a movable jaw of an end effector of a surgical instrument in accordance with an illustrative embodiment of this disclosure;

FIG. 23 is a bottom view of a cutting electrode assembled into a movable jaw of an end effector of a surgical instrument in accordance with an illustrative embodiment of this disclosure;

FIG. 24A is a front elevation, diagrammatic view of an exemplary patient side cart of a teleoperated surgical system;

FIG. 24B is a front elevation, diagrammatic view of an exemplary surgeon's console of a teleoperated surgical system;

FIG. 24C is a front elevation, diagrammatic view of an exemplary auxiliary control/vision cart of a teleoperated surgical system;

FIG. 25 is a perspective view of a teleoperated surgical instrument usable with an exemplary embodiment of the present teachings; and

FIG. 26 illustrates a perspective view of an illustrative surgical instrument with an end effector of the present disclosure.

### DETAILED DESCRIPTION

This description and the accompanying drawings illustrate exemplary embodiments and should not be taken as limiting, with the claims defining the scope of the present disclosure, including equivalents. Various mechanical, compositional, structural, and operational changes may be made without departing from the scope of this description and the claims, including equivalents. In some instances, well-known structures and techniques have not been shown or described in detail so as not to obscure the disclosure. Like numbers in two or more figures represent the same or similar elements. Furthermore, elements and their associated aspects that are described in detail with reference to one embodiment may, whenever practical, be included in other embodiments in which they are not specifically shown or described. For example, if an element is described in detail with reference to one embodiment and is not described with reference to a second embodiment, the element may nevertheless be claimed as included in the second embodiment. Moreover, the depictions herein are for illustrative purposes only and do not necessarily reflect the actual shape, size, or dimensions of the system or illustrated components.

It is noted that, as used in this specification and the appended claims, the singular forms “a,” “an,” and “the,” and any singular use of any word, include plural referents unless expressly and unequivocally limited to one referent. As used herein, the term “include” and its grammatical variants are intended to be non-limiting, such that recitation of items in a list is not to the exclusion of other like items that can be substituted or added to the listed items.

While the following disclosure is presented with respect to an end effector for a surgical instrument having two opposing jaws for clamping, dissecting and/or sealing tissue, it should be understood that the features of the presently described invention may be readily adapted for use in any type of surgical clamping, cutting, stapling or sealing instrument. For example, certain aspects of the presently described end effector may be employed in an surgical stapling instrument, such as any of those instruments described in commonly-assigned, co-pending U.S. Provisional Patent Application Nos. 62/947,307, 62/947,263 and 62/961,504; U.S. patent application Ser. Nos. 16/205,128, 16/678,405 and 16/904,482; and International Patent Nos. PCT/US2019/107646, PCT/US2019/019501, PCT/US2019/062344, PCT/US2019/064861, PCT/US2019/062768, PCT/2020/025655, PCT/US2020/056979, PCT/2019/066513, PCT/US2020/020672 and PCT/US2019/066530 and PCT/US2020/033481, the complete disclosures of which are incorporated by reference herein in their entirety for all purposes as if copied and pasted herein.

An end effector for a surgical instrument in accordance with embodiments of the present disclosure includes first and second jaws configured to grasp tissue therebetween. In certain embodiments, at least one of the jaws includes a sealing electrode on a tissue contacting surface thereof for applying energy to tissue grasped between the jaws, and a cutting electrode to dissect tissue previously (or simultaneously) sealed by the sealing electrodes. The cutting electrode may extend beyond the tissue contacting surface of the jaw and into a slot on the other jaw. In certain aspects of the invention, the cutting electrode is part of an electrode assembly that includes structure to permanently mount the electrode assembly to the jaw. In other aspects of the invention, mechanical components of the instrument, such as the actuation mechanisms for opening and closing the jaws, the articulation mechanisms for articulating the jaws relative the shaft and others, provide conductive pathways to supply electrosurgical energy to the sealing and cutting electrodes.

Surgical instruments of the present disclosure are adapted to be used with a robotic system for treating tissue with electrosurgical energy (e.g., cutting, sealing, ablating, etc.). The surgical instruments will generally include an actuation mechanism that controls the orientation and movement of the end effector. The actuation mechanism will typically be controlled by a robotic manipulator assembly that is controlled remotely by a user. For example, in one configuration, the actuation mechanism will be manipulated by the robotic manipulator assembly to move the jaws of the end effector between an open position and a closed position. In the closed position, the end effector will contact the electrodes against the tissue to cauterize and/or sever the engaged tissue. While described herein with respect to an instrument configured for use with a robotic surgical system, it should be understood that the end effectors and other structures of the surgical instruments described herein may be incorporated into manually actuated instruments, electro-mechanical powered instruments, or instruments actuated in any other way.

The sealing electrodes disposed on the end effector are contacted against the tissue so that current will flow from one electrode to the other electrode through the engaged tissue. In some configurations, the electrodes will both be disposed on the same end effector. When the end effector is in the closed position, the electrodes will be offset and spaced from each other such that delivery of a high frequency electrical energy will flow through the tissue between the electrodes without shorting the electrodes. Even if no tissue is between the end effector, there will typically be a gap between the electrodes. When the jaws of the end effector are in the closed position, the spacing between the negative and positive electrodes will generally be from about 0.01 and 0.10 inches, and in embodiments from about 0.010 inches and 0.025 inches. It should be appreciated however, that the spacing of the electrodes will vary depending on the area, volume, width, material of the electrodes, and the like. Similarly, electrode spacing and geometry may be adjusted to accommodate target tissue properties such as tissue thickness and impedance.

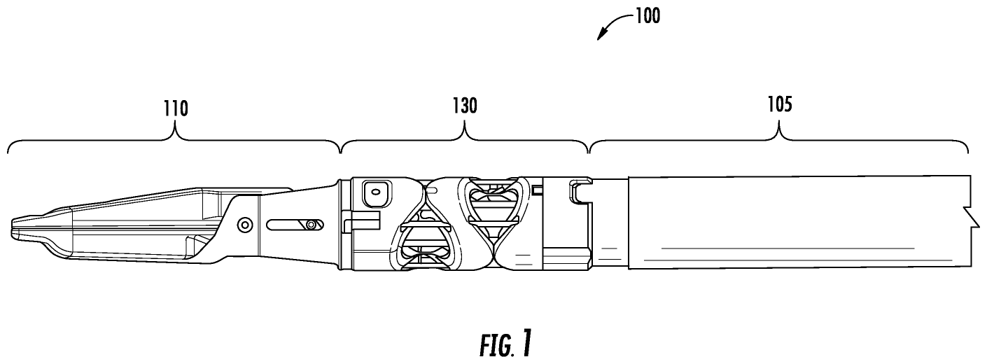

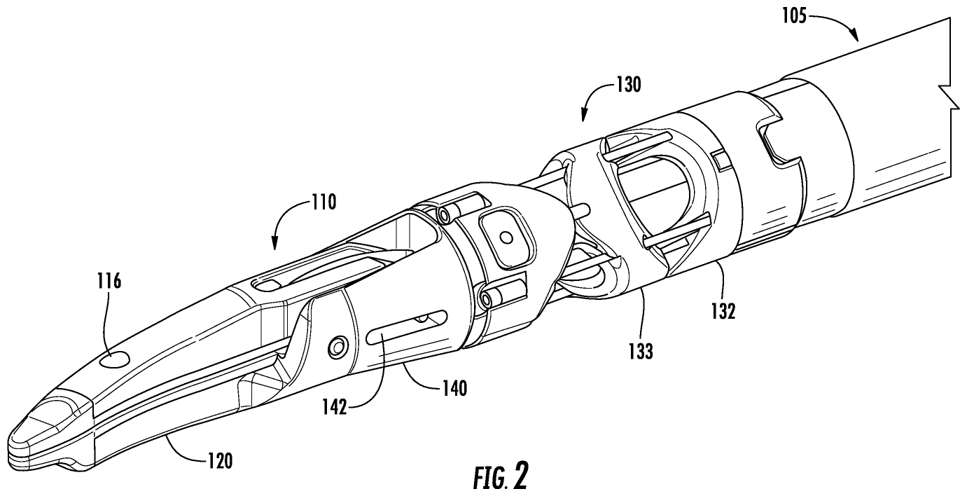

FIGS. 1 and 2 illustrate the distal end portion of a surgical instrument 100 in accordance with certain embodiments of the present disclosure. Surgical instrument 100 includes an end effector 110, an articulation mechanism 130, and an elongated shaft 105. The proximal end portion of elongate shaft 105 is operatively connected to an actuation mechanism (not shown), although as those skilled in the art reading this disclosure will appreciate, components of the actuation mechanism may extend into, and/or pass through elongated shaft 105 and/or articulation mechanism 130.

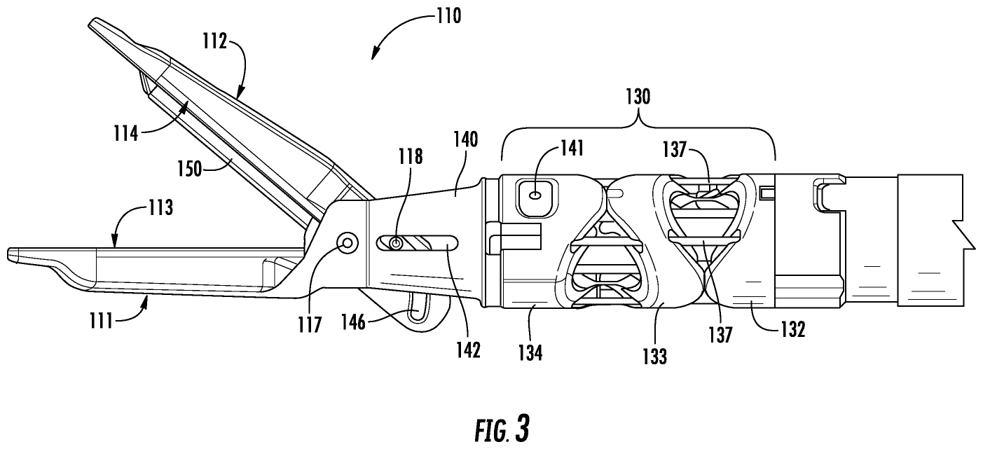

With reference now to FIG. 3, end effector 110 includes a first jaw 112, and a second jaw 111 configured to move between an open position (as shown in FIG. 3) where the jaws are spaced apart from one another and a closed position (shown in FIGS. 1 and 2) where tissue contacting surfaces 113, 114 cooperate to grasp tissue therebetween. First jaw 112 is a movable jaw configured to move from an open position to a closed position relative to second jaw 11. In other embodiments, first jaw 112 is a movable jaw configured to move between open and closed positions relative to second jaw 111. In still other embodiments, both jaws 112, 111 are movable relative to each other. In the exemplary embodiment shown, second jaw 11 is stationary and first jaw 112 is movable relative to second jaw 111 to pivot jaws 111, 112 between a fully open position, wherein the jaws define a desired angle between each other, to a substantially closed position, wherein the jaws are substantially parallel with each other.

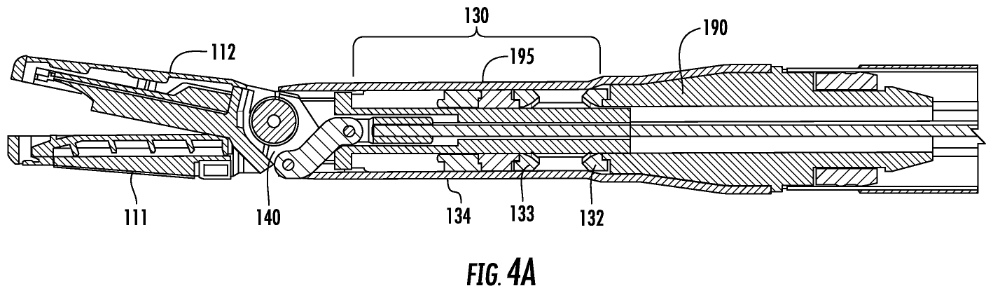

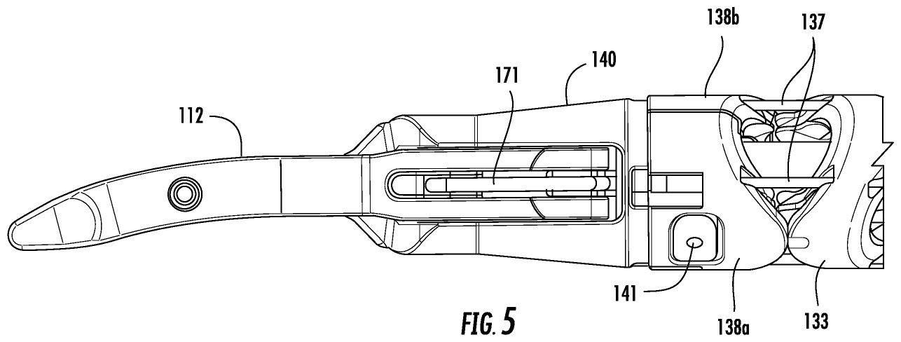

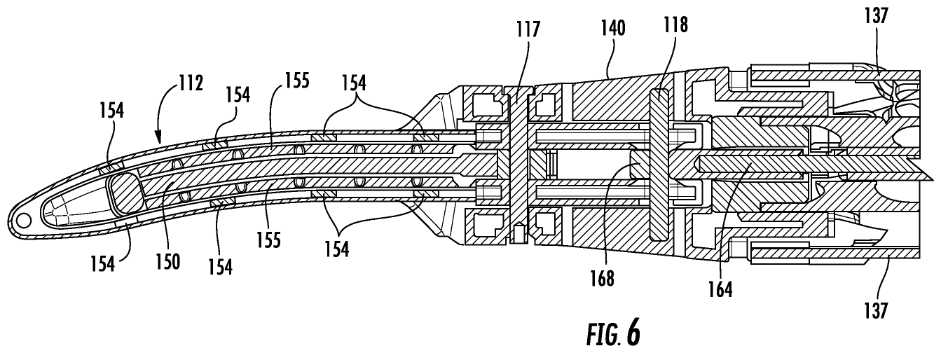

Stationary jaw 111 and movable jaw 112 further include sealing electrode(s) 155 (shown in FIG. 6) on tissue contacting surfaces 113, 114 for coagulating tissue grasped between jaws 111, 112. In an exemplary embodiment, jaws 111, 112 each include a sealing electrode 155 that is disposed on both sides of the longitudinal axis of end effector 110. However, it will be understood that other configurations are possible. For example, jaws 111, 112 may each include two or more sealing electrodes.

A cutting electrode 150 extends along a portion of movable jaw 112 and beyond tissue-contacting surface 114 for delivering high frequency electrical energy to sever grasped tissue. Stationary jaw 111 includes a central slot (not shown) into which cutting electrode 150 may extend when the jaws 111, 112 are in the closed position. Separate electrical pathways are provided to feed electrical current to each of the sealing electrode(s) 155 on stationary jaw 111, the sealing electrode(s) 155 on movable jaw 112, and the cutting electrode 150 on movable jaw 112.

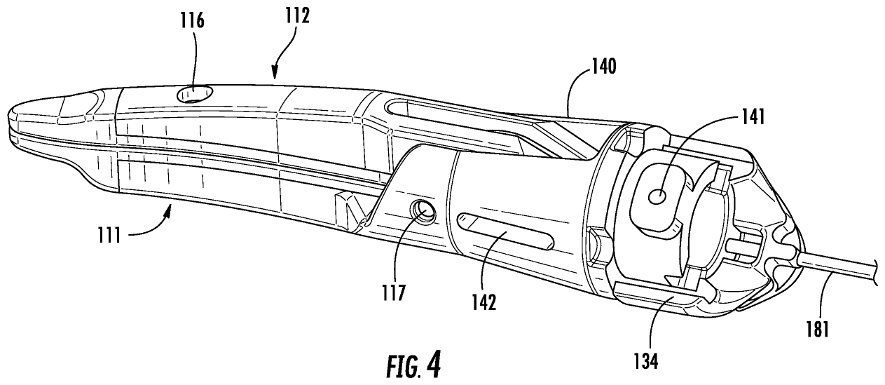

Stationary jaw 111 includes a clevis 140 at a proximal portion thereof as shown in FIGS. 3 and 4. A longitudinal slot 142 is formed on clevis 140. Slot 142 is substantially linear and substantially parallel to the tissue contacting surface 113 of stationary jaw 111. Movable jaw 112 includes a cam slot 146 (see FIG. 3). Cam slot 146 may be straight, curved, or may include a compound configuration in which a portion is straight and a portion is curved to achieve a desired mechanical advantage along different portions of a jaw opening and closing mechanism. A cam pin 118 travels along longitudinal slot 142 and within cam slot 146 such that proximal and distal translation of pin 118 pivots movable jaw 112 between the open and closed position.

Movement of jaws 111, 112 between the open and closed position is achieved by an actuation mechanism including a drive element (such as drive assembly 160 described below in connection with FIG. 8). In such a “push/pull” design, a compression/tension element may be used to move the end effector component. Pulling (tension) is used to move the component in one direction, and pushing (compression) is used to move the component in the opposite direction. In some implementations, the tension force (pulling) is used to actuate the end effector component in the direction that requires the highest force (e.g., closing jaws). The length of slot 142 defines the length of the gripping/actuation motion of a given surgical instrument. Cam pin 118 is operatively coupled to a drive element (see e.g., FIG. 6), and rides through slots 142 and 146 upon actuation, transitioning jaws 111, 112 between the open and closed positions as they pivot around a pivot pin 117. In the exemplary embodiment, as cam pin 118 is pulled in the proximal direction, jaws 111, 112 pivot towards the closed position to grasp tissue.

Surgical instruments in accordance with this disclosure may employ drive cables that are used in conjunction with a system of motors and pulleys. Powered surgical systems, including robotic surgical systems that utilize drive cables connected to a system of motors and pulleys for various functions including opening and closing of jaws, as well as for movement and actuation of end effectors are well known. Further details of known drive cable surgical systems are described, for example, in U.S. Pat. Nos. 7,666,191 and 9,050,119 both of which are hereby incorporated herein by reference in their entireties.

FIG. 3 shows a side view of end effector 110 and articulation mechanism 130 (e.g., wrist assembly 130) positioned between clevis 140 and elongated shaft 105. Wrist assembly 130, which may include wrist halves 138a, 138b, may provide a desired amount of motion, such as +/−90 degrees in a pitch or yaw direction. Wrist assembly 130 includes a proximal link 132, a middle link 133, and a distal link 134 that collectively determine the kinematic pitch and yaw motion of the wrist assembly 130. As shown, the interface between the proximal link 132 and the middle link 133 defines a joint that affects yaw movement of wrist assembly 130. The interface between the distal link 134 and the middle link 133 defines a joint that determines pitch movement of wrist assembly 130. However, in an alternative embodiment of a wrist assembly, this relationship can be reversed such that wrist assembly 130 pitches between proximal link 132 and middle link 133 and yaws between distal link 134 and middle link 133. Distal link 134 includes distal link halves that may be welded or otherwise secured to each other and rigidly attached to and provide mechanical connection and electrical isolation of wrist assembly 130 to clevis 140 of stationary jaw 111 via post 141. Cables 137 are drivingly coupled with the wrist assembly 130 and actuated to impart motion to wrist assembly 130. Differential movement of cables 137 can be used to actuate wrist assembly 130 to pitch and yaw at various angles. Additional details of articulation mechanisms usable with the embodiments disclosed herein are disclosed in Int'l. Pub. No. WO 2015/127250A1 and U.S. Publication No. 2017/0215977 A1, the entire disclosure of each of which is incorporated by reference herein.

FIG. 6 illustrates a cross-sectional view of the components of end effector 110, wrist assembly 130, and a portion of elongate shaft 105 shown in FIG. 2. To provide electrical current to surgical instruments in accordance with this disclosure, three conductive pathways may be provided, one for each of the sealing electrodes 155 contained on jaws 111, 112, and a third connection for cutting electrode 150. A first conductive pathway extends through a substantially central portion of a drive assembly 160 to provide electrical current to the sealing electrode(s) formed on movable jaw 112 (shown in FIG. 8), a second conductive pathway provides electrical current to cutting electrode 150 on movable jaw 112, and a third conductive pathway provides electrical current to the sealing electrode(s) on stationary jaw 111. These three conductive pathways will be further described below.

FIG. 8 illustrates drive assembly 160 which not only makes up part of the drive mechanism that opens and closes jaws 111, 112, but also serves as an electrical pathway to provide current to the sealing electrode(s) 155 on movable jaw 112. A wire 161 extends from a proximal end of the drive assembly 160 and is configured for connection to a power source (not shown). Wire 161 also extends through a drive tube 163 and along a central portion of drive assembly 160 in a distal direction. A distal end of wire 161 attaches to a drive rod 164. A yoke 168 is crimped to the distal end of drive rod 164.

As shown in FIG. 8, yoke 168 has an opening 165 formed thereon to receive cam pin 118, such that upon actuation, the forces provided by the actuation mechanism may be applied via yoke 168 to cam pin 118 to translate cam pin 118 as described above to open and close the jaws. Because drive rod 164 and yoke 168 of drive assembly 160 are conductive, and cam pin 118 is also conductive, the electrical current from wire 161 ultimately travels through these components to energize movable jaw 112, and specifically, sealing electrode 155 on movable jaw 112. Cam slot 146 is conductive and electrically coupled to sealing electrode 155 of movable jaw 112 such that electric current may pass from cam pin 118 to sealing electrode 155. An insulative coating on slot 142 electrically isolates cam pin 118 from stationary jaw 111.

In certain embodiments, cam slot 146 has conductive surfaces and insulating surfaces (not shown). The conductive surfaces transmit electric current from cam pin 118 to the sealing electrodes on movable jaw 112. The insulating surfaces ensure that the electric current is isolated from other components of the instrument. In an exemplary embodiment, a proximal portion of cam slot 146 is conductive and a distal portion is insulative. In this embodiment, the electric current is not transmitted to the sealing electrodes until cam pin 118 is positioned in the proximal portion of cam slot 146 (i.e., when the jaws are fully or partially closed). This ensures that the sealing electrodes cannot be energized when the jaws are fully open.

Drive assembly 160 further includes an insulation layer 166 disposed over a distal portion of drive tube 163 and a portion of drive rod 164. Insulation layer 166 provides additional strain relief, abrasion resistance, and environmental protection within drive assembly 160 and provides electrical isolation between drive assembly 160 and other electrically conductive portions of the instrument. Insulation layer 166 may be any appropriate material to achieve the desired properties. In embodiments, insulation layer 166 may be a heat shrink material, such as, for example, polytetrafluoroethylene (PTFE). PTFE provides for a low coefficient of friction, high temperature resistance, high shrink ratios, high dielectric strength, and is suited for extrusion into desired wall sections that are sufficiently thin for a given surgical instrument. One of ordinary skill in the art reading this disclosure will appreciate that the movable jaw 112 and the conductive components described above are preferably insulated and electrically isolated from stationary jaw 111.

FIG. 6 illustrates a bottom cross-sectional view of movable jaw 112. Movable jaw 112 is attached to stationary jaw 111 by means of pivot pin 117. Although components of drive assembly 160 are conductive, as described above, in embodiments all non-tissue contacting surfaces of movable jaw 112 are covered with an electric insulator. Suitable electric insulator materials may any medical grade material that provides high dielectric strength as well as arc track and flame resistance, including various rubber silicones, or plastics. Insulative layers on movable jaw 112 and on stationary jaw 111 prevent electrical connectivity between the two jaws via cam pin 118 (which, as noted above, is conductive).

Jaws 111, 112 may include insulating spacers 154 extending downwards and upwards from stationary jaw 111 and/or movable jaw 112, to prevent sealing electrodes 155 on each jaw from contacting each other and shorting when the jaws 112, 112 are in the closed position. In certain embodiments, spacers 154 are made from insulative materials such as ceramics, alumina, plastics, or silicone rubber. Spacers 154 may be arranged in any desirable configuration including parallel strips of material, increasing or decreasing in size along the length of the jaws, or any other desirable shape or configuration as may be beneficial for specific tissue thickness or a specific procedure.

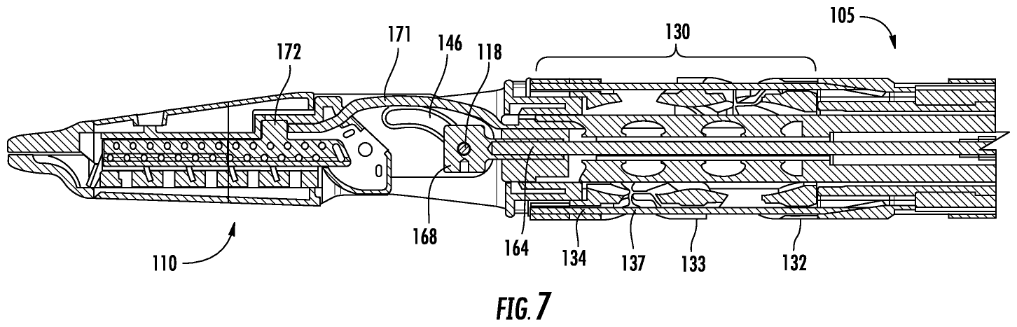

FIG. 7 illustrates the conductive electrical connection to provide current to cutting electrode 150 on movable jaw 112. A wire 171 provides electrical current solely to cutting electrode 150. Wire 171 extends through wrist assembly 130 and ultimately connects to a tab 172 of cutting electrode 150 and thus is located above the tissue contacting surface of moveable jaw 112 and above the majority of cutting electrode 150.

To provide electrosurgical energy to sealing electrode 155 on stationary jaw 111, wire 181 runs through wrist assembly 130 and is conductively connected to clevis 140 (see FIG. 4). Because stationary jaw 111 is rigidly attached via clevis 140 to wrist assembly 130, a mechanical connection to the rest of wrist assembly 130 is possible while maintaining electrical isolation. A proximal end of wire 181 is connected to a generator (not shown) that provides electrical current, and wire 181 in turn provides electrical current for sealing electrode 155 of stationary jaw 111 as it runs from a generator (not shown), through wrist assembly 130 and connects to clevis 140 of stationary jaw 111. As with movable jaw 112, in embodiments, non-tissue-contacting surfaces of stationary jaw 111 are covered with electrical insulator materials such as plastic, coatings, or combinations thereof to provide for further electrical isolation.

In an alternative embodiment, as shown in FIG. 4A, the diameter of surgical instrument 100 may be reduced by eliminating the presence of wire 181 within wrist assembly 130. In the embodiment of FIG. 4A, wire 181 contacts a proximal portion 191 of a tube adapter 190. Tube adapter 190 may be formed of any desired conductive metal. Tube adapter 190 is mechanically and electrically coupled with metal wrist links 132, 133, and 134, which are mechanically and electrically coupled to clevis 140. Clevis 140 is mechanically and electrically coupled with stationary jaw 111, thereby providing current to sealing electrode 155 of stationary jaw 111. In this embodiment, because wrist assembly 130 is conductive and energized, a wrist sheath 195 surrounds wrist assembly 130. Wrist sheath 195 may be made from similar materials and perform similar functions as insulation layer 166 described above.

FIG. 7 shows a cross-sectional side view of stationary jaw 111 and movable jaw 112 in the closed position to grasp and sever tissue. In use, cutting electrode 150 contained in movable jaw 112 cuts tissue by creating a high energy-density surface. The high energy density creates heat, which vaporizes tissue in contact with cutting electrode 150, resulting in tissue being transected along a cutting surface of cut electrode 150. Cutting electrode 150 is preferably located down a midline of movable jaw 112, as best seen in FIG. 6, and is flanked by sealing electrodes 155 on either side of movable jaw 112 and by sealing electrodes 155 on the stationary jaw 111. As a result, in operation, tissue is coagulated on either side of cut electrode 150, and is cut down the middle in between the two regions of coagulation. Because cutting electrode 150 is energized by a separate wire than sealing electrodes 155, cutting electrode 150 may be activated after activation of sealing electrodes 155, thereby ensuring the tissue is sealed before being cut. Cutting electrode 150 must withstand high heat, be electrically isolated from sealing electrodes 155, and avoid potential damage from other rigid objects that may be in close proximity, such as other instruments, staples, etc.

In some embodiments, cutting and sealing may occur at substantially the same time. In other embodiments, cutting may occur after the seal has been created on either side of the line of tissue dissection. This can be accomplished manually by the user through suitable controls on the proximal end of the instrument (or via a robotic control system). Alternatively, the control system may be designed to prevent the cutting electrode from being energized for a period of time after the sealing electrodes have been energized (i.e., a few second or a sufficient period of time to complete a tissue seal on either side of the line of tissue dissection).

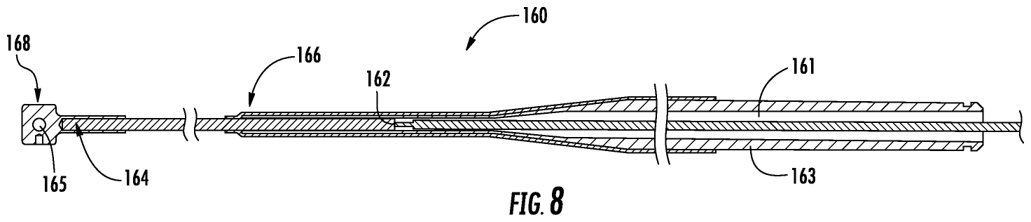

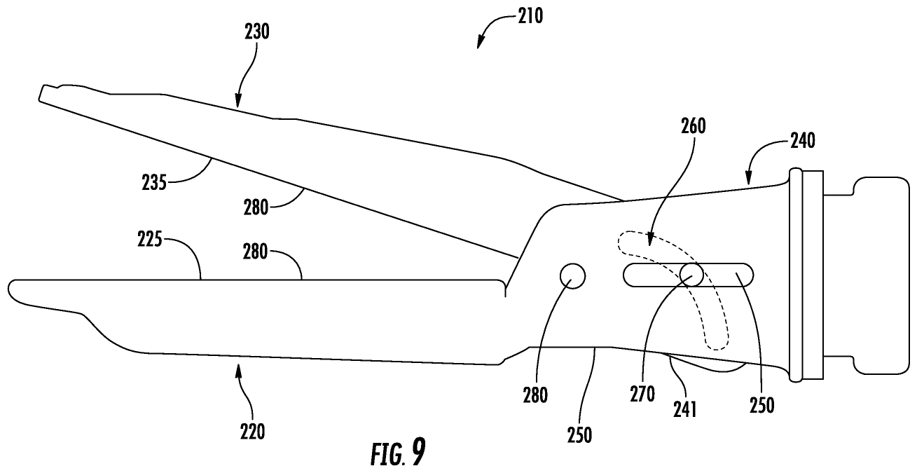

Referring now to FIG. 9, an end effector 210 for a surgical instrument, such as the illustrative surgical instrument shown in FIGS. 1-8, will now be described. As shown, end effector 210 includes first and second jaws 220, 230 which may be attached to a surgical instrument via a clevis 240. Clevis 240 further includes an opening for receiving a pivot pin 280 defining a pivot axis around which jaws 220, 230 pivot, as described in more detail below. A more complete description of a suitable clevis 240 for use with the present invention may be found in commonly-assigned, co-pending provisional patent application numbers: 62/783,444, filed Dec. 21, 2018; 62/783,481, filed Dec. 21, 2018; 62/783,460, filed Dec. 21, 2018; 62/747,912, filed Oct. 19, 2018; and 62/783,429, filed Dec. 21, 2018, the complete disclosures of which are hereby incorporated by reference in their entirety for all purposes. Of course, it will be recognized by those skilled in the art that other coupling mechanisms known by those skilled in the art may be used with the present invention to attach the jaws 220, 230 to the proximal portion of a surgical instrument.

In certain embodiments, first jaw 220 is a movable jaw configured to move from an open position to a closed position relative to second jaw 230. In other embodiments, first jaw 220 is a movable jaw configured to move between open and closed positions relative to second jaw 230. In still other embodiments, both jaws 220, 230 are movable relative to each other. In the exemplary embodiment shown, first jaw 220 is stationary and second jaw 230 is movable relative to first jaw 220 to pivot jaws 220, 230 between a fully open position, wherein the jaws define a desired angle between each other, to a substantially closed position, wherein the jaws are substantially parallel with each other.

According to one embodiment of the present disclosure, first jaw 220 comprise a substantially linear cam slot 250, and second jaw 230 comprises a non-linear cam slot 260. A cam slot pin 270 is disposed within cam slots 250, 260 and configured to translate distally and proximally therethrough. Distal translation of cam slot pin 270 causes second jaw 230 to close relative to first jaw 220 and proximal translation of cam slot pin 270 causes the jaws 220, 230 to open.

In an exemplary embodiment, cam slot 260 is curved and preferably shaped such that second jaw 230 applies a grip force against first jaw 220 that is substantially proportional to a force applied to cam slot pin 270 to translate pin 270 through slots 250, 260 (i.e., the ratio between the force input and the resulting force output remains substantially the same as pin 270 travels through the entire length of slots 250, 260). This design provides a constant mechanical advantage between the force applied to cam slot pin 270 and the force applied by jaws 220, 230 to tissue held therebetween, thereby allowing a user (or a robotic system) to more easily regulate the forces applied to tissue by jaws 220, 230.

In addition, this design allows for a substantially constant grip force to be applied by jaws 220, 230 regardless of the angle between jaws 220, 230. Therefore, at least in certain embodiments, 220, 230 jaws may apply substantially the same amount of grip force against, for example, a larger vessel or tissue portion that requires jaws 220, 230 to remain further open (e.g., from 100% of the fully open position down to 20% of the fully open position) than the force jaws 220, 230 would apply in a more closed position (i.e., less than 20% of the fully open position).

In an exemplary embodiment, non-linear slot 260 is configured and dimensioned to provide a constant mechanical advantage to end effector 210 as cam slot pin 270 moves throughout the length of slot 260 and the gripping/actuation motion. Applicant has discovered a critical profile for cam slot 260 that will provide this constant mechanical advantage throughout substantially the entire range of motion of jaws 220, 230. Assuming there is relatively low friction compared to driving forces, (which can be provided by the surface finish in cam slots 250, 260 and pin 270 travelling therethrough), and that the forces are transferred to the central axis of pin 270, the profile of cam slot 260 to provide for constant mechanical advantage may be determined using the following equation:

$$R ( θ ) = \frac{( a - b )}{λ} θ + b$$

wherein R is the cam slot profile as a function of jaw angle θ, a is the distance between distal pivot pin 280 and cam slot pin 270 when the jaws are in a fully open configuration, b is the distance between distal pivot pin 280 and cam slot pin 270 when the jaws are fully closed, λ is the maximum jaw angle when the jaws are 100% open, and θ is the instantaneous jaw angle having a range from 0 to λ.

A cam slot profile derived from this equation allows for greater control over forces exerted by the jaws during the entire ranging of motion of jaw opening and closing, as the force applied by the jaws will be a constant multiple of the force applied to the pin by the drive mechanism. For the majority of an instrument's range of motion, tissue handling is a high priority, and the foregoing configuration of cam slot 260 provides for constant mechanical advantage that allows a user to more easily regulate the forces being applied while grasping tissue.

Of course, it will be recognized by those of skill in the art that the present disclosure is not limited to the above embodiment. For example, cam slot 250 may be curved and cam slot 260 substantially linear. In this embodiment, cam slot 250 provides the substantially constant mechanical advantage to jaws 220, 230. In another configuration, both cam slots 250, 260 may be curved and shaped in combination to provide a substantially constant mechanical advantage to jaws 220, 230.

In certain embodiments, end effector 210 may further include electrodes 280 on one or both of the jaws in order to function as an electrosurgical instrument. In bipolar embodiments, electrodes 280 comprise tissue contacting surfaces 225, 235 on each of the jaws 220, 230. Electrodes 280 are then connected to output electrodes of electrical generators such that the opposing jaws are charged to different electrical potentials. Organic tissue, being electrically conductive, thereby allows for the two electrodes to apply electrical current through the grasped tissue in the closed position to heat tissue or blood vessels to cause coagulation or cauterization. For additional details on general aspects of electrosurgical instruments such as those described herein, see, e.g., U.S. Pat. No. 5,674,220, the entire disclosure of which is incorporated herein by reference for all purposes.

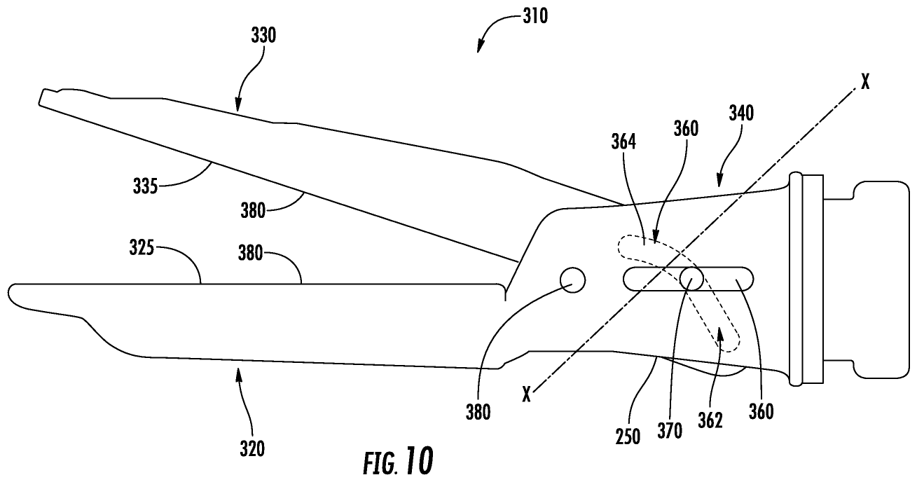

FIG. 10 illustrates another embodiment of an end effector 310 according to the present disclosure. Similar to the previous embodiment, end effector 310 includes first and second jaws 320, 330 which may be attached to a surgical instrument via a clevis 340. Clevis 340 further includes an opening for receiving a pivot pin 380 defining a pivot axis around which jaws 320, 330 pivot, as described in more detail below. In certain embodiments, first jaw 320 is a movable jaw configured to move from an open position to a closed position relative to second jaw 330. In other embodiments, first jaw 320 is a movable jaw configured to move between open and closed positions relative to second jaw 330. In still other embodiments, both jaws 320, 330 are movable relative to each other. In the exemplary embodiment shown, first jaw 320 is stationary and second jaw 330 is movable relative to first jaw 320 to pivot jaws 320, 330 between a fully open position, wherein the jaws define a desired angle between each other, to a substantially closed position, wherein the jaws are substantially parallel with each other.

First jaw 320 comprise a substantially linear cam slot 350, and second jaw 330 comprises a compound cam slot 360. A cam slot pin 370 is disposed within cam slots 350, 360 and configured to translate distally and proximally therethrough. Proximal translation of cam slot pin 370 causes second jaw 330 to close relative to first jaw 320 and distal translation of cam slot pin 370 causes the jaws 320, 330 to open.

Compound cam slot 360 comprises a non-linear distal portion 364 and a substantially linear proximal portion 362 (the junction between proximal portion 364 and distal portion 362 is indicated by dotted line X-X). Distal portion 364 is shaped such that jaws 320, 330 apply a substantially constant grip force therebetween as cam slot pin 370 is translated proximally through distal portion 364 (i.e., the force applied by movement of first jaw 330 is substantially proportional to the force applied to cam slot pin 370 as pin 370 is translated proximally through distal portion 364). Proximal portion 362 of compound slot 360 is shaped to provide a non-constant grip force between jaws 320, 330 as cam slot pin 370 is translated through proximal portion 362 (i.e., the force applied by movement of first jaw 330 increases non-proportionally relative to the force applied to can slot pin 370 as pin 370 is translated distally through proximal portion 362).

The curved distal portion 364 of compound slot 360 provides a substantially constant mechanical advantage when jaws 320, 330 are partially or substantially open. In this configuration, jaws 320, 330 are typically used to perform tasks, such as tissue handling. This allows the user to more easily regulate the forces being applied to tissue grasped between jaws 320, 330. In an exemplary embodiment, distal portion 364 has a profile to provide for constant mechanical advantage similar to, or the same as, the profile described above with respect to cam slot 260 in FIG. 4.

The substantially linear proximal portion 362 of compound slot 360 provides an elevated mechanical advantage as cam slot pin 370 travels through proximal portion 362 (i.e., jaws 320, 330 apply a stronger grip force as they close). In this configuration, jaws 320, 330 are typically being used for sealing vessels. Elevating the mechanical advantage between the input force (i.e., the force applied to pin 370) and output force (i.e., the forces applied by jaws 320, 330 to tissue) enhances tissue/vessel compression and seal.

Of course, it will be recognized that other configurations are possible. For example, cam slot 350 may be a compound slot while slot 360 is substantially linear. Alternatively, both cam slots 350, 360 may have curved proximal portions that operate in combination to provide a constant mechanical advantage to jaws 320, 330. In yet another embodiment, end effector 310 may include multiple cam slot pins. For example, a proximal cam slot pin may translate through a curved proximal cam slot in one of the jaws and a distal cam slot pin translate through a substantially linear cam slot. The proximal cam slot pin actuates jaws 320, 330 for a first portion of the actuation stroke and the distal cam slot pin actuates jaws 320, 330 for a second portion of the actuation stroke.

In an exemplary embodiment, first and second jaws 320, 330 define a first angle therebetween in the fully open position, and a second angle therebetween when cam slot pin 370 is located at a junction between distal and proximal portions 362, 364 of compound slot 360. The second angle is preferably about 50% or less the first angle, more preferably about 20% or less. Thus, proximal portion 362 of compound slot 360 corresponds with an angle of about 50% or less, preferably about 20% or less, of the total angle between jaws 320, 330 in the fully open configuration. For example, when jaws 320, 330 are at least 50% open (or at least 20% open in certain embodiments), cam slot pin 370 resides in the curved distal portion 364 of compound slot 360 and the force applied by jaws 320, 330 to tissue is substantially proportional to the force applied to cam slot pin 370 as pin 370 translates through distal portion 364. When jaws 320, 330 are less than 50% open (or less than 20% open in certain embodiments), cam slot pin 370 resides in the substantially linear proximal portion 362 of compound slot 360 and the force applied by jaws 320, 330 to tissue is non-proportional to the force applied to pin 370 (i.e. elevated mechanical advantage) as pin 370 translates through proximal portion 362.

In an exemplary embodiment, distal portion 364 is actively engaged by cam slot pin 370 within cam slot 360 when the jaws are between about 20% to about 100% open and proximal portion 362 is actively engaged by cam slot pin 370 within cam slot 360 when the jaws are between about 0% to about 20% open. Cam slot 360 has a point of inflection along an axis X-X, where cam slot 360 transitions from providing a constant mechanical advantage, to providing a non-constant mechanical advantage as cam slot pin 370 is translated from the distal portion 364 to the proximal portion 362.

The resulting compound cam slot 360 created by the combination of proximal portion 362 and distal portion 364 allows a user to have the control and benefits of constant mechanical force throughout a relatively large portion of the actuation stroke. Additionally, as the actuation stroke nears completion, a user benefits from the proximal portion 362 of the cam slot which is configured to provide a higher mechanical advantage to ensure that sufficient clamping force is achieved before the instrument's function is carried out, such as sealing, stapling, or other useful functions. For sealing, a compound cam slot in accordance with this disclosure may be configured to ensure that a user achieves operating pressures of about 3 kg/cm2 to about 16 kg/cm2 to effect a proper and effective tissue seal.

End effector 310 may further include an electrodes 390 on one or both of the jaws in order to function as an electrosurgical instrument. In bipolar embodiments, electrodes 390 may comprise tissue contacting surfaces 325,335 on each of the jaws 320,330.

The end effectors in accordance with the presently described embodiments may be readily adapted for use in any type of surgical clamping, cutting, and/or sealing instruments. For example, features of the present surgical instruments may be employed to treat tissue with electrosurgical energy (e.g., cutting, sealing, ablating, etc.). The surgical instrument including the present end effectors may be a minimally invasive (e.g., laparoscopic) instrument or an instrument used for open surgery.

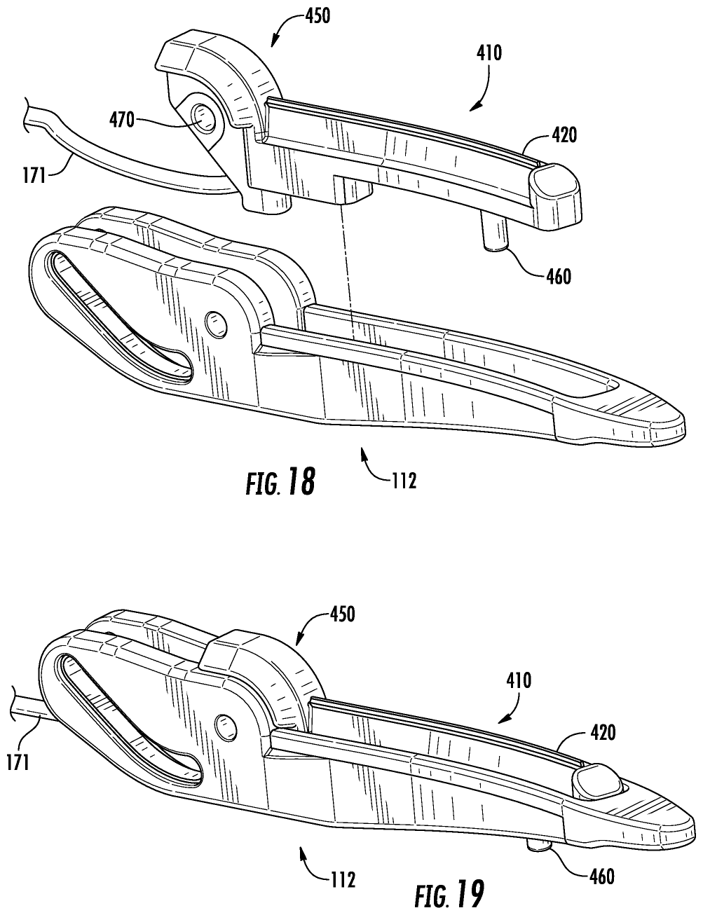

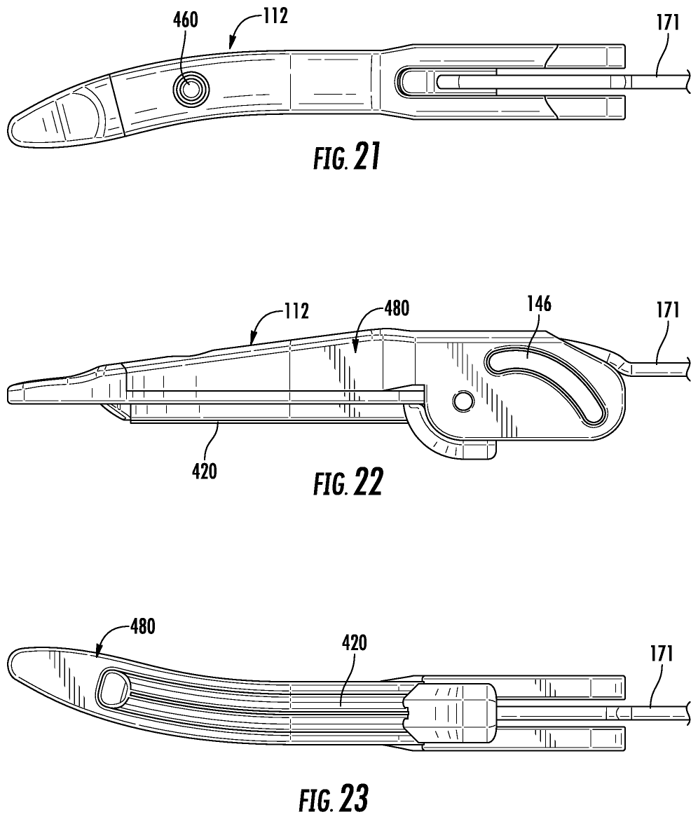

FIGS. 11-23 depict an illustrative method of manufacturing a cutting electrode assembly to support a cutting electrode 150 in accordance with embodiments of the present disclosure.

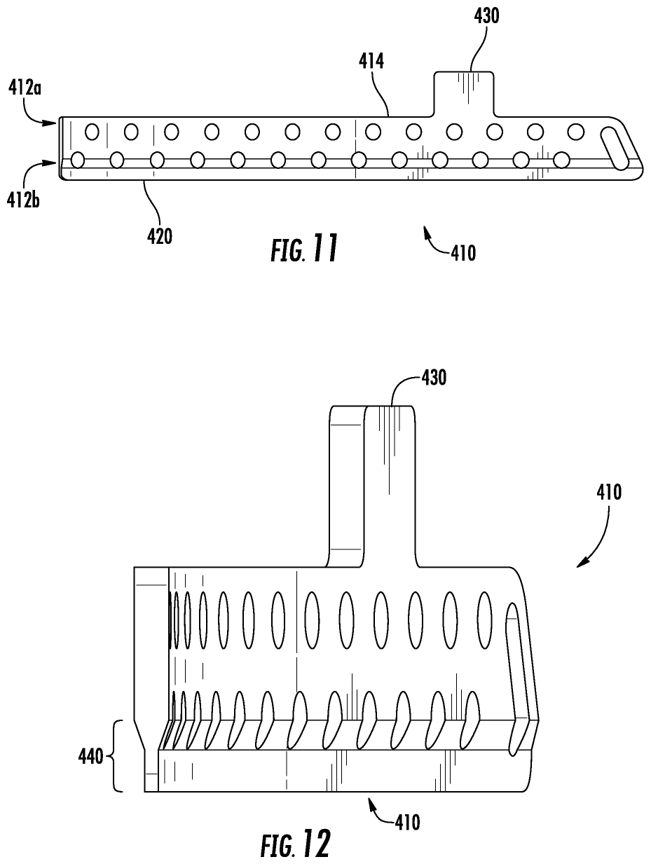

As shown in FIG. 11, a bare metal electrode 410 is initially shaped into a desired form. Electrode 410 may be made from any suitable conductive material such as, for example, aluminum, copper, silver, tin, gold, tungsten, platinum, or the like. In certain embodiments, electrode 410 is made of stainless-steel. Electrode 410 may include a tissue contacting portion (cutting surface) 420, two rows of openings 412a and 412b, and a tab 430 formed on a proximal portion of electrode 410. Tab 430 is positioned on edge 414 of electrode 410 opposite the tissue-contacting portion 420. Initially, electrode 410 is formed from stock material of a first thickness, which is then coined to form a thinner section 440 as shown in FIG. 12. This ensures the exposed portion of electrode 410 has a desired thickness, while simultaneously cold-working the material in order to increase rigidity. Electrode 410 is then ready to have a first layer of insulator applied thereto.

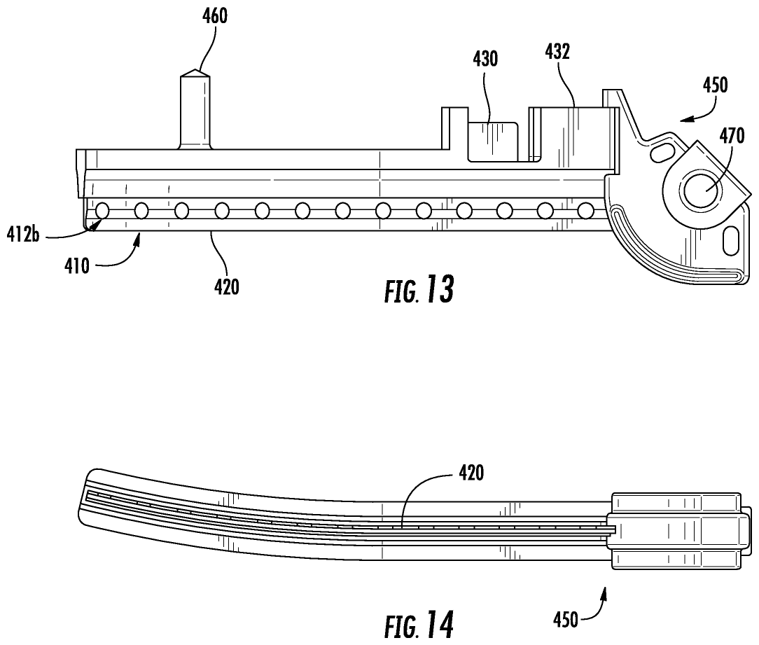

FIGS. 13-14 depict injection-molding a first-shot insulator layer 450 over electrode 410. Insulating layer 450 preferably comprises a material that has high temperature resistance, electrical isolation and sufficient rigidity to provide a stable mechanical attachment of the electrode to the first jaw, such as plastic, ceramic, or any other moldable insulating material. Other processes for forming first-shot insulator layer 450 over electrode 410 may include the use of deposition processes or the use of various securing or adhering means. Insulator layer 450 preferably fills the row of openings 412a to aid in securing insulator layer 450 to electrode 410.

Insulator layer 450 includes two distinct structures for attaching the finished cut electrode assembly to the jaws. First, insulator layer 450 includes an attachment structure for securing electrode 410 to jaws 111, 112. In an exemplary embodiment, the attachment structure comprises a post 460 which may be substantially aligned with tab 430 and allows for cutting electrode 410 to be secured to jaws 111, 112. Post 460 may be any other structure that functions to allow for cutting electrode 410 to be secured to jaws 111, 112. In an exemplary embodiment, post 460 is configured for passing through an opening 116 in jaw 112, as discussed in further detail below in relation to FIGS. 18A and 18B.

Second, the insulator layer 450 includes a pivot pin hole 470 for receiving pivot pin 417 upon assembly of electrode 410 with movable jaw 112. In addition to these two structures, insulator layer 450 includes a proximal tab 432, which functions to provide a structure for wire routing, wire strain-relief and electrical isolation between electrode 410 and the jaws 111, 112, while also providing rigidity and stability to the thin sheet metal assembly.

Referring now to FIGS. 15-16, a wire 171 is fed through proximal tab 432 of insulator layer 450 and then attached to electrode tab 430 by welding, soldering, or any other suitable attachment method. Wire 171 is connected to a power source (not shown) and generally runs through the wrist assembly 130 of the surgical instrument as previously described in connection with illustrative surgical instruments and end effectors discussed above. In embodiments, wire 171 may have a mass greater than bare metal electrode 410. It is therefore mechanically advantageous to have wire 171 run through components that are above, or out of alignment with, the remainder of electrode 410 in a way similar to proximal tab 432 and tab 430.

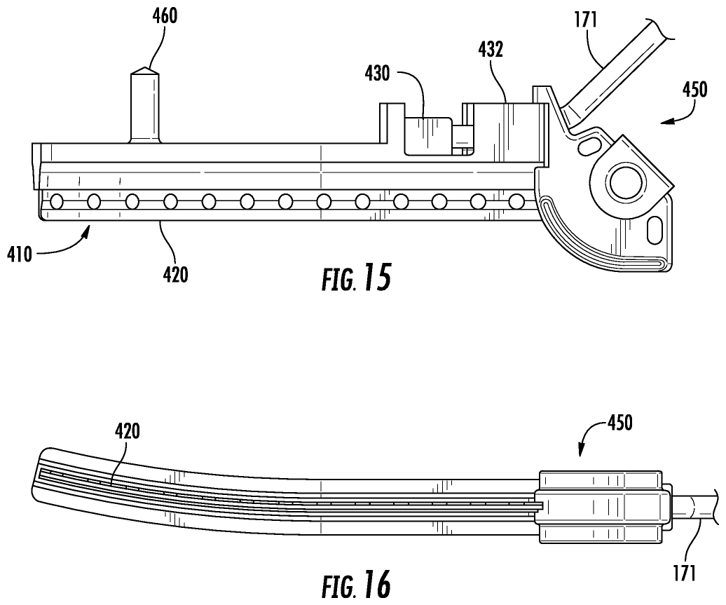

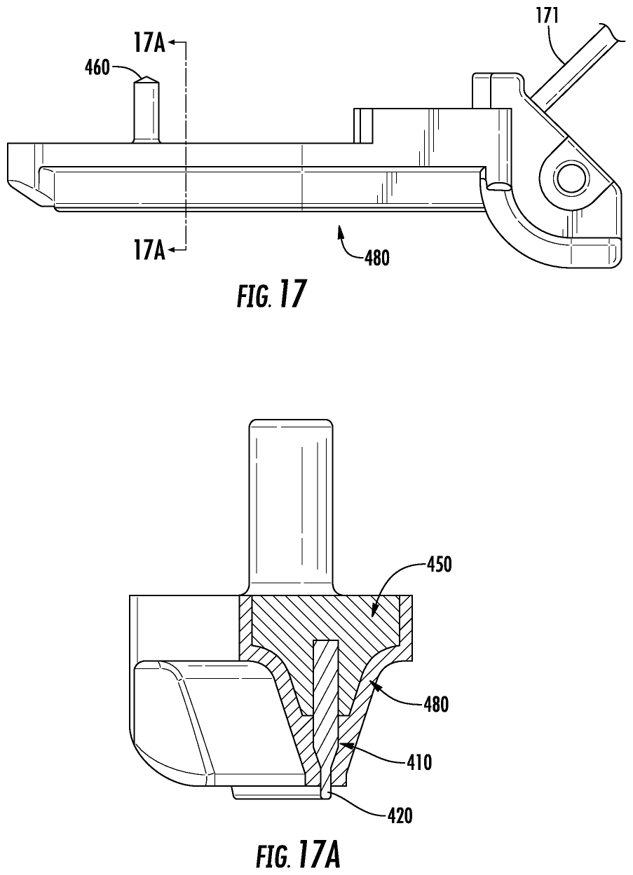

Subsequently, as shown in FIG. 17, a second shot of insulating material is applied to provide insulating layer 480. Insulator layer 480 may be made from silicone rubber or any other suitable material having sufficiently high dielectric strength and sufficiently high temperature resistance required to prevent damage to the insulation when cutting tissue. In embodiments, insulator layer 480 is hydrophobic, has non-stick properties, and has a relatively high comparative tracking index (CTI), to deter incidence of arc tracking from the high voltages required for cutting tissue. Insulator layer 480 fills row of openings 412b (see FIG. 11) to aid in securing insulator layer 480 to electrode 410. Insulator layer 480 allows for a thin layer of insulation to be deposited along either of side adjacent the tissue contacting surface 420 of electrode 410, thereby only allowing a small area of the cut electrode 410 to be exposed to the target tissue. Insulator layer 480 also acts as a potting material over the site of connection of wire 171 to tab 430 to insulate wire 171 and tab 430 from the rest of the instrument. After the application of insulator layer 480 is completed, the cut assembly is prepared for attachment to the jaws.

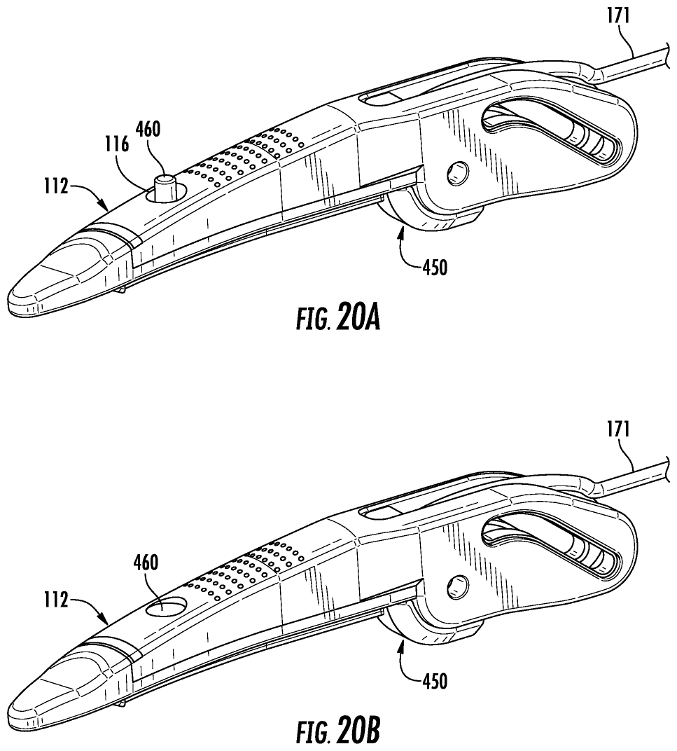

FIGS. 18-20 illustrate installation of a finished cut electrode assembly into the jaws. Once the cut electrode assembly is installed into jaw 112, it is then secured in place. To secure cutting electrode 410 to jaw 112, post 460 is positioned within opening 116 on jaw 112 and is deformed such that post 460 is secured to opening 116. Post 460 may be deformed through a variety of different methods, including cold forming or thermoplastic staking. In an exemplary embodiment, post 460 is heat staked to the opposing side of the jaw 112 (as best seen in FIGS. 20A and 20B). This permanently attaches cutting electrode 410 to jaw 112 and does not allow further movement of the cutting electrode 410 with respect to jaw 112. Additionally, a pivot pin 117 is then inserted through a pivot pin hole 470, allowing movable jaw 112 to move simultaneously with the cutting electrode 410 relative to stationary jaw 111. FIGS. 21-23 illustrate various views of the cut electrode assembly fully installed on the jaw 112.

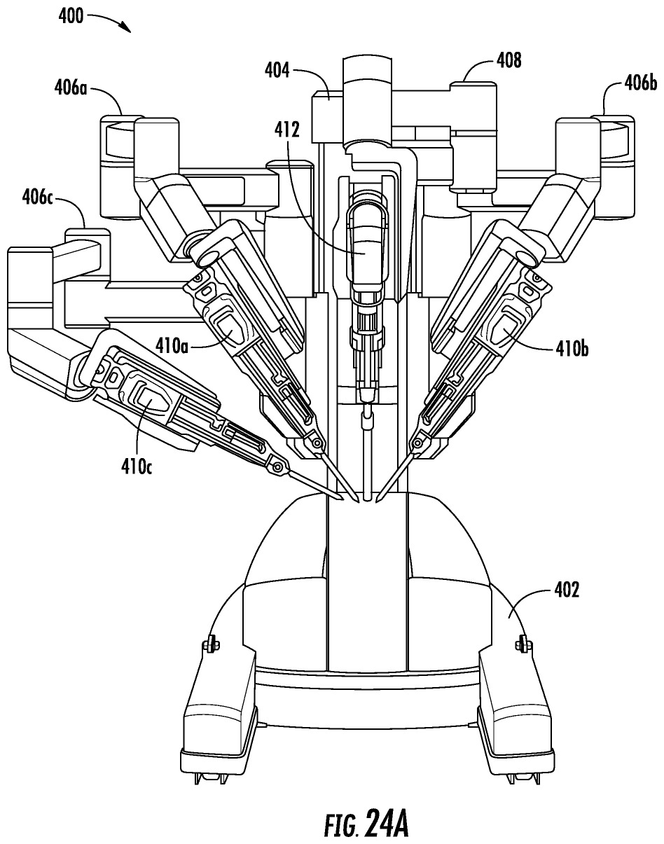

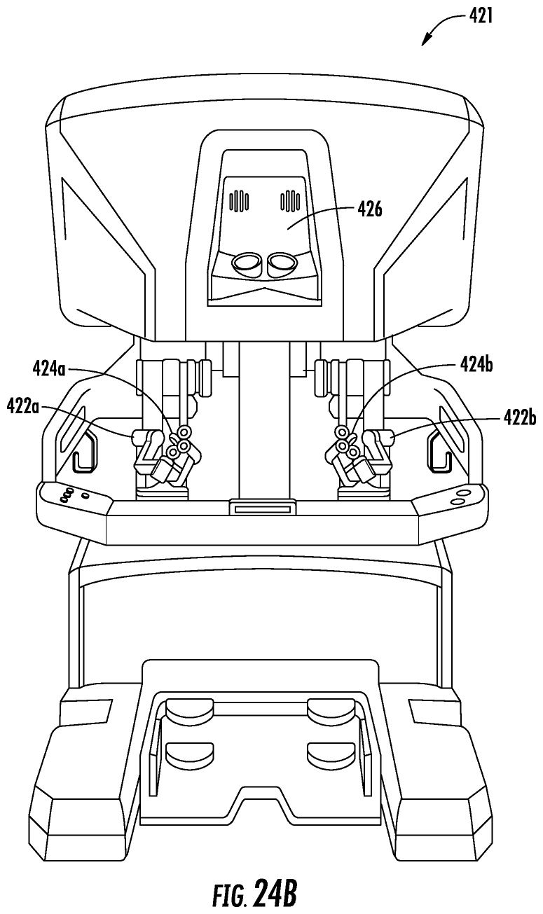

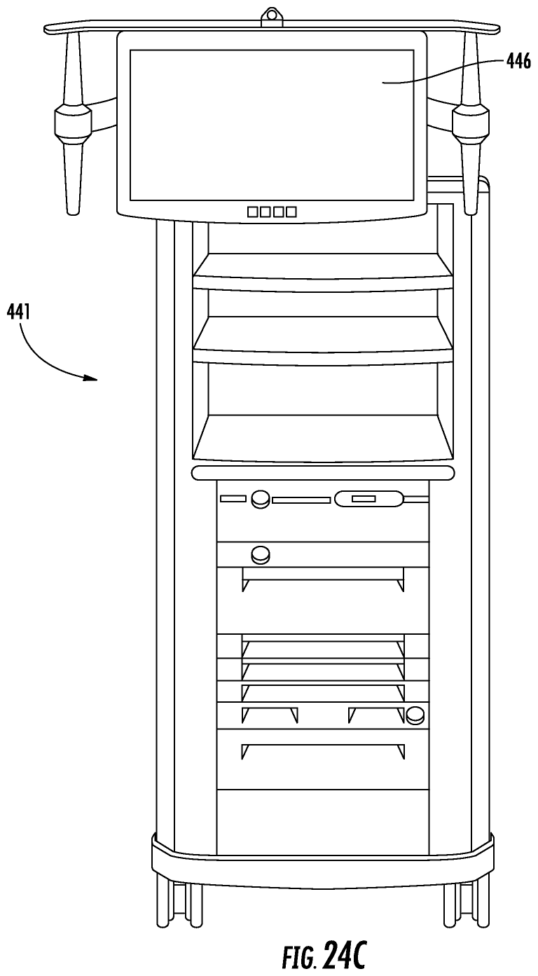

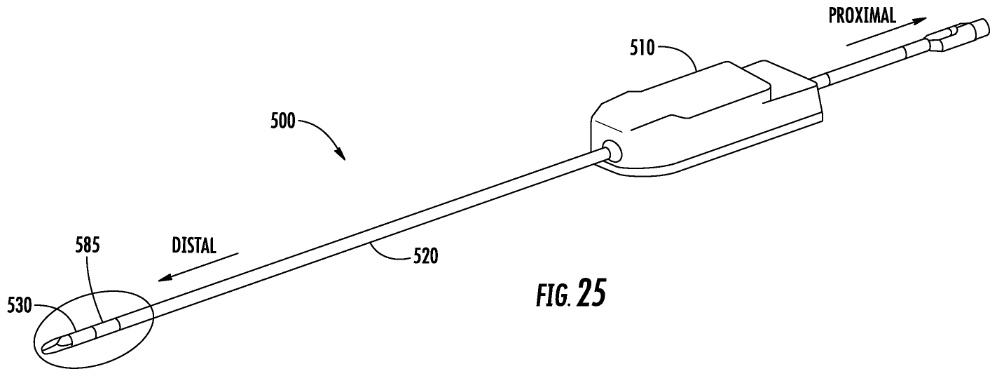

In certain embodiments, the end effectors described above in accordance with this disclosure may be used with surgical instruments incorporated into a robotic surgical system. FIGS. 24A, 24B, and 24C are front elevation views of three exemplary embodiments of main components of a teleoperated surgical system for minimally invasive surgery that may be used in combination with end effectors of the present disclosure. These three components are interconnected so as to allow a surgeon, for example, with the assistance of a surgical team, to perform diagnostic and corrective surgical procedures on a patient. In an exemplary embodiment, a teleoperated surgical system in accordance with the present disclosure may be embodied as a da Vinci® surgical system commercialized by Intuitive Surgical, Inc. of Sunnyvale, Calif. Also, for a further explanation of a teleoperated surgical system, including a patient side cart, surgeon's console, and auxiliary control/vision cart, with which the present disclosure may be implemented, reference is made to U.S. Patent App. Pub. No. 2011/0071542A1, which is incorporated by reference in its entirety herein. However, the present disclosure is not limited to any particular surgical system, and one having ordinary skill in the art reading this disclosure would appreciate that the disclosure herein may be applied in a variety of surgical applications, including other teleoperated surgical systems.

FIG. 24A is a front elevation view of an exemplary embodiment of a patient side cart 100 of a teleoperated surgical system. The patient side cart 400 includes a base 402 that rests on the floor, a support tower 404 mounted on the base 402, and one or more manipulator arms mounted on the support tower 404 and that support surgical instruments and/or vision instruments (e.g., a stereoscopic endoscope). As shown in FIG. 24A, manipulator arms 406a, 406b are arms that support, and transmit forces to manipulate, the surgical instruments used to grasp and move tissue, and arm 408 is a camera arm that supports and moves the endoscope. FIG. 24A also shows a third manipulator arm 406c that is supported on the back side of support tower 404 and that is positionable to either the left or right side of the patient side cart as desired to conduct a surgical procedure.

Interchangeable surgical instruments 410a, 410b, 410c can be installed on the manipulator arms 406a, 406b, 406c, and an endoscope 412 can be installed on the camera arm 108. Those of ordinary skill in the art reading this disclosure will appreciate that the arms that support the instruments and the camera may also be supported by a base platform (fixed or moveable) mounted to a ceiling or wall, or in some instances to another piece of equipment in the operating room (e.g., the operating table). Likewise, they will appreciate that two or more separate bases may be used (e.g., one base supporting each arm).

Control of the robotic surgical system, including control of the surgical instruments, may be effectuated in a variety of ways, depending on the degree of control desired, the size of the surgical assembly, and other factors. In some embodiments, the control system includes one or more manually operated input devices, such as a joystick, an exoskeletal glove, pincher or grasper assemblies, buttons, pedals, or the like. These input devices control servo motors which, in turn, control the articulation of the surgical assembly. The forces generated by the servo motors are transferred via drivetrain mechanisms, which transmit the forces from the servo motors generated outside the patient's body through an intermediate portion of the elongate surgical instrument 110 to a portion of the surgical instrument inside the patients body distal from the servo motor.

FIG. 24B is a front elevation view of an exemplary surgeon's console 421 of a teleoperated surgical system for controlling the insertion and articulation of surgical instruments. The surgeon or other system operator manipulates input devices by moving and repositioning input devices within console 421. As illustrated in the exemplary embodiment of FIG. 24B, the surgeon's console is equipped with master controllers or master input devices. As illustrated in FIG. 24B, master input devices may include left and right multiple degree-of-freedom (DOF) master tool manipulators (MTM's) 422a, 422b, which are kinematic chains that are used to control the surgical tools (which include the endoscope and various cannulas mounted on arms 406, 408 of the patient side cart 400). Each MTM may include an area for surgeon or operator input. For example, as shown in FIG. 24B, each MTM 422a, 422b may include a pincher assembly 424a, 424b. The surgeon grasps a pincher assembly 424a, 424b on each MTM 422a, 422b, typically with the thumb and forefinger, and can move the pincher assembly to various positions and orientations. When a tool control mode is selected, each MTM 422 is coupled to control a corresponding manipulator arm 406 for the patient side cart 400, as those of ordinary skill in the art are familiar. The pincher assembly is typically used to operate a surgical end effector (e.g., scissors, grasping retractor, needle driver, hook, forceps, spatula, etc.) at the distal end of an instrument 410.

The surgeon's console 421 also can include an image display system 426. In an exemplary embodiment, the image display is a stereoscopic display wherein left side and right side images captured by the stereoscopic endoscope 412 are output on corresponding left and right displays, which the surgeon perceives as a three-dimensional image on display system 426.

The surgeon's console 421 is typically located in the same operating room as the patient side cart 400, although it is positioned so that the surgeon operating the console may be outside the sterile field. One or more assistants may assist the surgeon by working within the sterile surgical field (e.g., to change tools on the patient side cart, to perform manual retraction, etc.). Accordingly, the surgeon may operate remote from the sterile field, and so the console may be located in a separate room or building from the operating room. In some implementations, two consoles 421 (either co-located or remote from one another) may be networked together so that two surgeons can simultaneously view and control tools at the surgical site.

For additional details on the construction and operation of general aspects of a teleoperated surgical system such as described herein, see, e.g., U.S. Pat. Nos. 6,493,608 and 6,671,581, the entire disclosure of each of which is incorporated herein by reference.

As shown in FIG. 24C, the auxiliary control/vision cart 441 includes an optional display 446 (e.g., a touchscreen monitor), which may be mounted elsewhere, such as on the patient side cart 400. The auxiliary control/vision cart 441 further includes space 448 for optional auxiliary surgical equipment, such as electrosurgical units, insufflators, and/or other flux supply and control units. The patient side cart 400 (FIG. 6A) and the surgeon's console 421 (FIG. 6B) are coupled via optical fiber communications links to the auxiliary control/vision cart 441 so that the three components together act as a single teleoperated minimally invasive surgical system that provides an intuitive telepresence for the surgeon.

In accordance with various exemplary embodiments, the present disclosure contemplates controlling a surgical instrument such that a gripping force applied by an end effector of the instrument is substantially linear throughout a range of motion of the end effector for a given force applied to a push-pull (drive) rod of the instrument to actuate the end effector.

With reference to FIG. 25, an exemplary embodiment of a teleoperated surgical instrument 500 that may support a previously described end effector of the present disclosure is depicted. As shown, surgical instrument 500 generally includes a housing 510 at its proximal end. Housing 510 may include an instrument memory or storage device (not shown). The memory can perform a number of functions when the instrument is loaded on the manipulator arm 406. For example, the memory can provide a signal verifying that the instrument is compatible with that particular surgical system. Additionally, the memory may identify the instrument and end effector type (whether it is a scalpel, a needle grasper, jaws, scissors, a clip applier, an electrocautery blade, or the like) to the surgical system so that the system can reconfigure its programming to take full advantage of the instrument's specialized capabilities. As further discussed below, the memory may include specifics on the architecture of the instrument, and include particular values that should be employed in control algorithms, such as tool compliance and gain values.

Housing 510 also may include a force/torque drive transmission mechanism (not shown) for receiving output from motors of the manipulator arm 406, the force/torque drive transmission mechanism transmitting the output from the motors to an end effector 530 of the instrument through an instrument shaft 520 mounted to the transmission mechanism. Exemplary surgical robotic instruments, instrument/manipulator arm interface structures, and data transfer between the instruments and servomechanism is more fully described in U.S. Pat. No. 6,331,181, the full disclosure of which is incorporated herein by reference.

Surgical instrument 500 comprises an end effector 530 disposed at the distal end of an elongate shaft 520 and may be connected thereto by a clevis 585 that supports and mounts end effector 530 relative to instrument shaft 520. As embodied herein, shaft 520 may be a relatively flexible structure that can bend and curve. Alternatively, shaft 520 may be a relatively rigid structure that does not permit traversing through curved structures. Optionally, in some embodiments, instrument 500 also can include a multi-DOF articulable wrist structure (not shown) that supports end effector 530 and permits multi-DOF movement of the end effector in arbitrary pitch and yaw. Those having ordinary skill in the art are familiar with a variety of wrist structures used to permit multi-DOF movement of a surgical instrument end effector.

For additional details on robotic surgical systems, see, e.g., commonly owned U.S. Pat. No. 6,493,608 “Aspects of a Control System of a Minimally Invasive Surgical Apparatus,” and commonly owned U.S. Pat. No. 6,671,581 “Camera Referenced Control in a Minimally Invasive Surgical Apparatus,” which are hereby incorporated herein by reference in their entirety for all purposes. A more complete description of illustrative robotic surgical systems for use with the present invention can be found in commonly-assigned U.S. Pat. Nos. 9,295,524, 9,339,344, 9,358,074, and 9,452,019, the complete disclosures of which are hereby incorporated by reference in their entirety for all purposes.

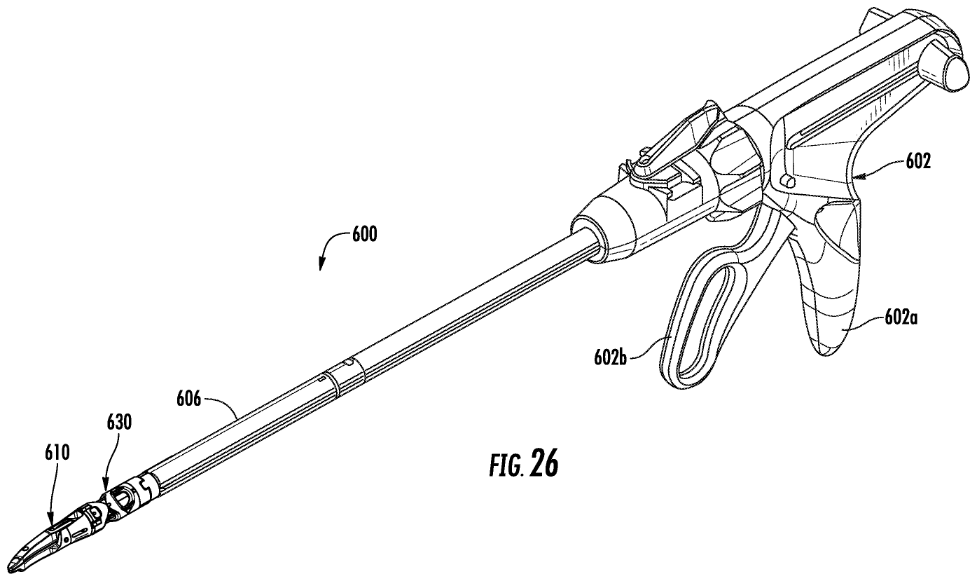

FIG. 26 is a perspective view of another illustrative surgical instrument 600 that may incorporate the end effectors described above in accordance with certain embodiments of the present disclosure. As shown, surgical instrument 600 includes a handle assembly 602, and an end effector 610 mounted on an elongated shaft 606 of the surgical stapling instrument 600. End effector 610 includes a first jaw 611 and a second jaw 612. Handle assembly 602 includes a stationary handle 602a and a moveable handle 602b, which serves as an actuator for surgical instrument 600.

In certain embodiments, handle assembly 602 may include input couplers (not shown) instead of, or in addition to, the stationary and movable handles. The input couplers provide a mechanical coupling between the drive tendons or cables of the instrument and motorized axes of the mechanical interface of a drive system. The input couplers may interface with, and be driven by, corresponding output couplers (not shown) of a telesurgical surgery system, such as the system disclosed in U.S. Pub. No. 2014/0183244A1, the entire disclosure of which is incorporated by reference herein for all purposes. The input couplers are drivingly coupled with one or more input members (not shown) that are disposed within the instrument shaft 606 and end effector 610. Suitable input couplers can be adapted to mate with various types of motor packs (not shown), such as the stapler-specific motor packs disclosed in U.S. Pat. No. 8,912,746, or the universal motor packs disclosed in U.S. Pat. No. 8,529,582, the disclosures of both of which are incorporated by reference herein in their entirety for all purposes. Further details of known input couplers and surgical systems are described, for example, in U.S. Pat. Nos. 8,597,280, 7,048,745, and 10,016,244. Each of these patents is hereby incorporated by reference in its entirety for all purposes.

Actuation mechanisms of surgical instrument 600 may employ drive cables that are used in conjunction with a system of motors and pulleys. Powered surgical systems, including robotic surgical systems that utilize drive cables connected to a system of motors and pulleys for various functions including opening and closing of jaws, as well as for movement and actuation of end effectors are well known. Further details of known drive cable surgical systems are described, for example, in U.S. Pat. Nos. 7,666,191 and 9,050,119 both of which are hereby incorporated by reference in their entireties for all purposes. While described herein with respect to an instrument configured for use with a robotic surgical system, it should be understood that the wrist assemblies described herein may be incorporated into manually actuated instruments, electro-mechanical powered instruments, or instruments actuated in any other way.

Hereby, all issued patents, published patent applications, and non-patent publications that are mentioned in this specification are herein incorporated by reference in their entirety for all purposes, to the same extent as if each individual issued patent, published patent application, or non-patent publication were specifically and individually indicated to be incorporated by reference.

While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of presently disclosed embodiments. Thus the scope of the embodiments should be determined by the appended claims and their legal equivalents, rather than by the examples given.

Persons skilled in the art will understand that the devices and methods specifically described herein and illustrated in the accompanying drawings are non-limiting exemplary embodiments. The features illustrated or described in connection with one exemplary embodiment may be combined with the features of other embodiments. Various alternatives and modifications can be devised by those skilled in the art without departing from the disclosure. Accordingly, the present disclosure is intended to embrace all such alternatives, modifications and variances. As well, one skilled in the art will appreciate further features and advantages of the present disclosure based on the above-described embodiments. Accordingly, the present disclosure is not to be limited by what has been particularly shown and described, except as indicated by the appended claims.

What is claimed is:

1. A surgical instrument comprising:
first and second jaws movable relative to each other between open and closed positions;
an electrode coupled to the first jaw;
an actuator mechanism coupled to the first and second jaws and configured to move the jaws between the open and closed positions; and
wherein at least one portion of the actuator mechanism comprises a conductive pathway for electrically coupling the electrode to a source of energy.

2. The surgical instrument of claim 1, wherein the actuator mechanism comprises a slot in the first jaw and a pin positioned within the slot such that translation of the pin through the slot rotates the jaws between the open and closed positions.

3. The surgical instrument of claim 1, wherein the pin comprises a conductive material and is configured for electrical coupling to the source of energy.

4. The surgical instrument of claim 3, wherein at least one surface of the slot comprises a conductive material such that contact between the pin and said at least one surface electrically couples the electrode to the source of energy.

5. The surgical instrument of claim 4, wherein the actuator mechanism further comprises a drive rod for translating the pin through the slot, wherein the drive rod comprises a conductive pathway electrically coupling the pin with the source of energy.

6. The surgical instrument of claim 5, wherein the slot is a first slot and further comprising a second slot in the second jaw, wherein the pin is positioned within the second slot such that translation of the pin through the first and second slots rotates the jaws between the open and closed positions, and further comprising an insulating material between the pin and the second slot to electrically isolate the pin from the second jaw.

7. The surgical instrument of claim 1, further comprising an elongate shaft and a coupling member coupling the elongate shaft with the first and second jaws, wherein the coupling member includes a conductive member configured for coupling to the source of energy.

8. The surgical instrument of claim 7, further comprising a second electrode on the second jaw, wherein the second electrode is electrically coupled to the conductive member within the coupling member.

9. The surgical instrument of claim 8, wherein the coupling member comprises an articulation mechanism configured to articulate the first and second jaws relative to the elongate shaft.

10. The surgical instrument of claim 9, wherein the articulation mechanism comprises a wrist assembly, wherein the wrist assembly comprises a conductive material, the instrument further comprising an insulating sheath disposed around the wrist assembly.

11. The surgical instrument of claim 1, wherein the actuator mechanism is coupled to a control device of a robotic surgical system.

12. A surgical instrument comprising:
first and second jaws movable relative to each other between open and closed positions;
an electrode having a cutting surface;
a first insulating layer covering a first portion of the electrode and having an attachment structure for attaching the electrode to the first jaw; and
a second insulating layer covering a second portion of the electrode such that the cutting surface remains exposed.

13. The surgical instrument of claim 12, wherein the first jaw comprises an opening and the attachment structure comprises a post extending from the first insulating layer through the opening.

14. The surgical instrument of claim 13, wherein the post is deformed to secure the post within the opening of the first jaw.

15. The surgical instrument of claim 14, wherein the post is heat staked to the opening in the first jaw.

16. The surgical instrument of claim 12, wherein the electrode comprises a first row of holes, the first insulating layer extending through the first row of holes.

17. The surgical instrument of claim 16, wherein the electrode comprises a second row of holes, the second insulating layer extending through the second row of holes.

18. The surgical instrument of claim 12, wherein the electrode further comprises a tab on an opposite surface as the cutting surface, the tab being configured to receive a wire to create a conductive junction between the electrode and a power source.

19. The surgical instrument of claim 12, wherein the first jaw and the first insulating layer each comprise a pivot hole for receiving a pivot pin therethrough, wherein the pivot pin is configured to allow the first jaw to pivot relative to the second jaw.

20. The surgical instrument of claim 25, wherein the electrode is a cutting electrode configured for dissecting tissue, the first jaw further comprising one or more sealing electrodes configured for sealing tissue.

21. The surgical instrument of claim 20, wherein the second jaw comprises one or more sealing electrodes configured for sealing tissue and wherein the first jaw comprises one or more spacers extending from the first jaw towards the second jaw to space the sealing electrodes on the first jaw from the sealing electrodes on the second jaw when the first and second jaws are in the closed position.
